(19) United States
(12) Patent Application Publication
(10) Pub. No.: US 2007/0123572 A1
(43) Pub. Date: May 31, 2007

(54) NOVEL METHOD OF PREPARATION OF 5-CHLORO-3-IMIDAZOL-1-YL-[1,2,4]THIADIAZOLE AND (3-IMIDAZOL-1-YL-[1,2,4]THIADIAZOL-5YL)-DIALKYL-AMINES

(75) Inventors: Mark R. Herbert, San Diego, CA (US); Russell D. Cousins, San Diego, CA (US); Hengyuan Lang, San Diego, CA (US); Timothy C. Gahman, San Diego, CA (US); Stewart A. Noble, San Diego, CA (US)

Correspondence Address: INTERNATIONAL PATENT GROUP;ATTN: MS LAVERN HALL, P.O. BOX 38129, ST. LOUIS, MO, 63138, US

(73) Assignee: KALYPSYS, INC., San Diego, CA (US)

(21) Appl. No.: 11/563,200

(22) Filed: Nov. 25, 2006

Related U.S. Application Data
(60) Provisional application No. 60/740,325, filed on Nov. 28, 2005.

(51) Int. Cl. A61K 31/433; C07D 417/04

(52) U.S. Cl. 514/362; 548/128

(57) ABSTRACT

The present invention discloses a novel method for preparing 3-imidazol-1-yl-[1,2,4]thiadiazole derivatives, particularly to a method of preparing 5-halo-3-imidazol-1-yl-[1,2,4]thiadiazole, more particularly (3 -imidazol-1-yl-[1,2,4]thiadiazol-5-yl)-dialkyl-amines, that afford a high yield of pure product.

This application claims the benefit of priority of U.S. provisional application No. 60/740,325, filed Nov. 28, 2005, the disclosure of which is hereby incorporated by reference as if written herein in its entirety.

### FIELD OF THE INVENTION

This invention relates to a method for preparing substituted 3-imidazol-1-yl-[1,2,4]thiadiazoles, particularly to a method of preparing 5-halo-3-imidazol-1-yl-[1,2,4]thiadiazole, and more particularly (3-imidazol-1-yl-[1,2,4]thiadiazol-5-yl)-dialkyl-amines, that afford a high yield of pure product.

### BACKGROUND OF THE INVENTION

It has been found that (3-imidazol-1-yl-[1,2,4]thiadiazol-5-yl)-dialkyl-amines may be obtained in a method wherein a 3-amino-5-halo-[1,2,4]thiadiazole is converted to a 5-halo-3-imidazol-1-yl-[1,2,4]thiadiazole through a cyclization and dehydration procedure then coupled to the appropriate amine, and an alternate method wherein a 2,5-dihalo-[1,2,4]thiadiazole is coupled to an appropriate amine and sodium imidazole. Novel intermediates generated in said method are also disclosed herein.

### SUMMARY OF THE INVENTION

This invention is directed to a novel, high yield method for preparing 3-imidazol-1-yl-[1,2,4]thiadiazoles, particularly to a method of preparing 5-halo-3-imidazol-1-yl-[1,2,4]thiadiazoles, more particularly (3-imidazol-1-yl-[1,2,4]thiadiazol-5-yl)-dialkyl-amines. According to the method disclosed herein, a 3-amino-5-halo-[1,2,4]thiadiazole is converted to a 5-halo-3-imidazol-1-yl-[1,2,4]thiadiazole through a cyclization and dehydration procedure, then coupled to the appropriate amine; or by an alternate method, a 2,5-dihalo-[1,2,4]thiadiazole is sequentially coupled to an appropriate amine and then to sodium imidazole.

### DETAILED DESCRIPTION OF THE INVENTION

This invention relates to a novel method for preparing in high yield (3-imidazol-1-yl-[1,2,4]thiadiazol-5-yl)-dialkyl-amines of structural formula (I): 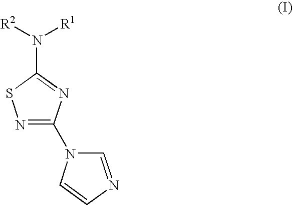
wherein:

R¹ and R² each represent any chemically reasonable substituent. For example, R¹ and R² may be independently selected from any of the following groups, or any combination of the following groups: hydrogen, acyl, alkanoyl, alkenyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted as defined herein; R¹ and R² may also be taken together to form a heterocycloalkyl ring. R¹ and R² may be optionally substituted as defined herein.

Said novel method is comprised of two independent routes, each having two reaction steps.

The first novel method comprises the following steps:

a) Treating a 3-amino-5-halo-[1,2,4]thiadiazole derivative of structural formula (II): 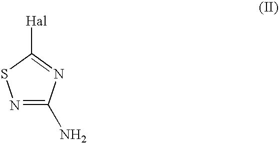
with a combination of suitable reagents, including formaldehyde, glyoxal, ammonium chloride and an appropriate amount of a protic acid in a suitable protic solvent, using an appropriate reaction time over a suitable temperature range as defined herein; with or without isolating the novel reaction product of structural formula (III); 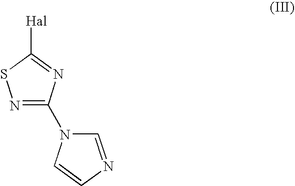
wherein:

Formaldehyde may be replaced with synthetic formaldehyde equivalents, including, but not limited to formaldehyde gas, formalin, formaldehyde sodium bisulfite addition product, paraformaldehyde, methylal, and s-1,3,5-trioxane;

Glyoxal may be used in any of it's several synthetic glyoxal equivalent forms, including but not limited to anhydrous glyoxal, glyoxal hydrate, and glyoxal bis-bisultite addition product forms;

Ammonium chloride may be replaced with synthetic ammonia equivalents, including, but not limited to ammonia gas, ammonium hydroxide, ammonium acetate, ammonium sulfate, ammonium bicarbonate, ammonium sulfate, and ammonium carbamate;

Suitable protic acids include but are not limited to phosphoric acid, hydrochloric acid, sulfuric acid, camphorsulfonic acid, benzenesulfonic acid, and p-toluenesulfonic acid. Said protic acids may be used in concentrations ranging from catalytic amounts (e.g. 0.001-0.1 equivalents), through stoichiometic amounts (e.g. 1 mole equivalent), through excess amounts (e.g. >1 mole equivalent);

Suitable protic solvents, which can optionally be combined together as co-solvents for this reaction step, include, but are not limited to ethanol, methanol, propanol, iso-propanol, butanol, tert-butanol, methoxyethanol, ethoxyethanol, ethylene glycol, propylene glycol and the like;

Suitable temperature ranges for this reaction step are from about −20° C. to 150° C., the preferred range being from about 40° C. to 90° C.; and

Suitable time periods for this reaction step range from 5 minutes to 48 hours, the preferred range being from about 16 to 20 hours.

then:

b) Reacting the reaction product of step a), the compound of structural formula (III), with an appropriate amine derivative R¹NHR², as defined below, in a suitable aprotic solvent; using an appropriate reaction time over a suitable temperature range as defined herein; and isolating the desired product of structural formula (I), in high purity;

wherein:

Suitable aprotic solvents, which can optionally be combined together as co-solvents for this reaction step, include, but are not limited to dichloromethane, DMSO, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H-pyrimidinone, hexamethylphosphoramide, acetonitrile, pyridine, 2,6-lutidine, 2,4,6-collidine, and the like;

Suitable temperature ranges for this reaction step are from about −70° C. to 100° C., the preferred range being from about −10° C. to 40° C.; and

Suitable time periods for this reaction step range from 5 minutes to 48 hours, the preferred range being from about 16 to 20 hours.

In certain embodiments, step a) comprises the reaction condition variation wherein excess of formalin, glyoxal, ammonium chloride, and an appropriate amount of a protic acid are combined and reacted in a suitable solvent using an appropriate reaction time over a suitable temperature range as defined herein, with or without isolating the novel reaction product of structural formula (III).

In certain embodiments, step a) comprises the reaction condition variation wherein stoichiometric amounts of formalin, glyoxal, ammonium chloride and a catalytic amount of phosphoric acid are combined and reacted in a suitable solvent using an appropriate reaction time over a suitable temperature range as defined herein, with or without isolating the novel reaction product of structural formula (III).

In certain embodiments, step a) comprises the reaction condition variation wherein an excess of paraformaldehyde, glyoxal, ammonium chloride, and an appropriate amount of protic acid are combined and reacted in a suitable solvent using an appropriate reaction time over a suitable temperature range as defined herein, with or without isolating the novel reaction product of structural formula (III).

In certain embodiments, step a) comprises the reaction condition variation wherein stoichiometric amounts of paraformaldehyde, glyoxal, ammonium chloride and a catalytic amount of phosphoric acid are combined and reacted in a suitable solvent using an appropriate reaction time over a suitable temperature range as defined herein, with or without isolating the novel reaction product of structural formula (III).

In certain embodiments, step a) comprises the reaction condition variation wherein an excess of methylal, glyoxal, ammonium chloride, and an appropriate amount of protic acid are combined and reacted in a suitable solvent using an appropriate reaction time over a suitable temperature range as defined herein, with or without isolating the novel reaction product of structural formula (III).

In certain embodiments, step a) comprises the reaction condition variation wherein stoichiometric amounts of methylal, glyoxal, ammonium chloride and a catalytic amount of phosphoric acid are combined and reacted in a suitable solvent using an appropriate reaction time over a suitable temperature range as defined herein, with or without isolating the novel reaction product of structural formula (III).

In certain embodiments, step a) comprises the reaction condition variation wherein an excess of s-1,3,5-trioxane, glyoxal, ammonium chloride, and an appropriate amount of protic acid are combined and reacted in a suitable solvent using an appropriate reaction time over a suitable temperature range as defined herein, with or without isolating the novel reaction product of structural formula (III).

In certain embodiments, step a) comprises the reaction condition variation wherein stoichiometric amounts of s-1,3,5-trioxane, glyoxal, ammonium chloride and a catalytic amount of phosphoric acid are combined and reacted in a suitable solvent using an appropriate reaction time over a suitable temperature range as defined herein, with or without isolating the novel reaction product of structural formula (III).

The second novel method comprises the following steps:

a) Treating a 3,5-dihalo[1,2,4]thiadiazole derivative of the structural formula (IV): 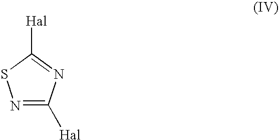
with an appropriate amine, as defined below, in a suitable aprotic solvent, using an appropriate reaction time over a suitable temperature range as defined herein; with or without isolating the novel reaction product, a 3-halo-1,2,4-thiadiazole-5-dialkyl amine derivative of structural formula (V): 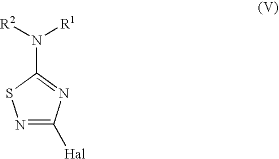
wherein:

Suitable aprotic solvents, which can optionally be combined together as co-solvents for this reaction step, include, but are not limited to dichloromethane, DMSO, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H-pyrimidinone, hexamethylphosphoramide, acetonitrile, pyridine, 2,6-lutidine, 2,4,6-collidine, and the like;

Suitable temperature ranges for this reaction step are from about −70° C. to 100° C., the preferred range being from about −10° C. to 40° C.; and

Suitable time periods for this reaction step range from 5 minutes to 48 hours, the preferred range being from about 16 to 20 hours;

then:

b) Reacting the product of step a), the compound of structural formula (V), with an imidazole or a 1,2,4-triazole salt derivative in a suitable dipolar aprotic solvent; using an appropriate reaction time over a suitable temperature range as defined herein, and isolating the desired product, in high purity;

wherein:

Suitable imidazole and 1,2,4-triazole salts include, but are not limited to sodium, potassium, lithium, and cesium salts of an optionally substituted imidazole or 1,2,4-triazole derivative;

Suitable dipolar aprotic solvents, which can optionally be combined together as co-solvents for this reaction step, include, but are not limited to DMSO, sulfolane, N,N-dimethylformamide, N,N-dimethylacetamide, 1,3-dimethyl-3,4,5,6-tetrahydro-2(1H-pyrimidinone, hexamethylphosphoramide, and the like;

Suitable temperature range for this reaction step is from about −10° C. to 200° C., the preferred range being from about 0° C. to 90° C.; and

Suitable time periods for this reaction step range from 5 minutes to 48 hours, the preferred range being about 8 to 16 hours.

The starting material of structural formula (II) may be prepared by standard methods known to those skilled in the art, by reacting a guanidine salt with trihalomethanesulfenyl halide in the presence of an alkali metal hydroxide and a suitable solvent.

It will be obvious to one skilled in the art that the Hal group in compound (II) may be chlorine, bromine, or iodine. Such a starting material may be carried through steps (a) and (b) of the method described above, to give a compound similar to that of formula (I).

One embodiment for preparing compounds of structural formula (I) comprises:
a) Reacting a compound of structural formula (II) with an excess amount of glyoxal, formaldehyde, ammonium chloride, and phosphoric acid in a suitable solvent, from about ambient temperature to 80° C., with vigorous mixing of reagents, and optionally isolating the novel compound of structural formula (III): 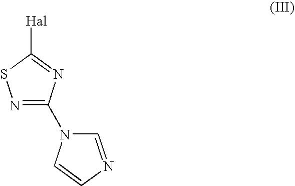
then
b) Reacting the compound of structural formula (III) with an appropriately protected dialkylamine and a tertiary amine base in a suitable solvent, over a temperature range of from about 0° C. to reflux for about 5 minutes to 48 hours and isolating the desired product.

Another embodiment is the method for preparing the compound of structural formula (I), comprising:
a) Reacting the compound of structural formula (II), wherein Hal=Cl, with an excess of glyoxal, formalin, ammonium chloride and phosphoric acid in a solution of ethanol, from about ambient temperature to 80° C., with vigorous mixing of reagents, with isolation of the reaction product of structural formula (IIIa); 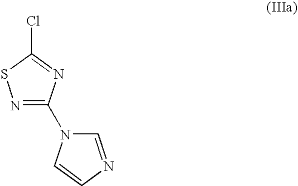
then:
b) Reacting the reaction product of structural formula (3a) with a dialkylamine derivative, R¹NHR², wherein R¹ and R² are as defined above, in the presence of triethylamine, in DMSO, at about ambient temperature for about 5 minutes to 48 hours, and isolating the desired product, in high yield and in a state of high purity.

Another embodiment of the method for preparing the compound of structural formula (I) comprises:
a) Reacting a compound of structural formula (IV) with a stoichiometric amount of a dialkylamine derivative, R¹NHR², wherein R¹ and R² are as defined above, or a metal salt thereof, in the presence of a tertiary amine base in a suitable solvent, at a temperature from about 0° C. to 30° C., over a time period from about 5 minutes to 48 hours, and optionally isolating the novel compound of structural formula (V); 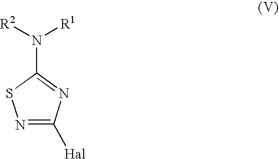
then:
b) Reacting the compound of structural formula (V) with a salt of imidazole in a suitable solvent, at about ambient temperature to 100° C., over a time period of about 5 minutes to 48 hours, and isolating the desired product.

A further embodiment of the present invention is the method for preparing the compound of structural formula (VI): 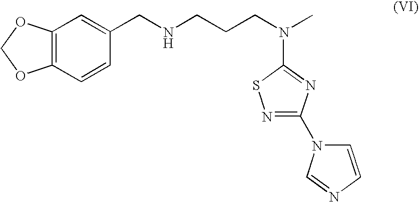
comprising:
a) reacting the compound of structural formula (IV), wherein Hal=C, with benzo[1,3]dioxol-5-ylmethyl-(3-methylamino-propyl)-carbamic acid tert-butyl ester in the presence of a trialkylamine in a suitable aprotic solvent, using an appropriate reaction time over a suitable temperature range, with or without isolating the reaction product of structural formula (VIa); 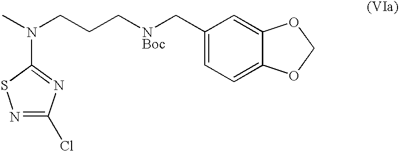
then:
b) reacting the compound of structural formula (VIa) with a imidazole salt in a suitable dipolar aprotic solvent, using an appropriate reaction time over a suitable temperature range, and isolating the desired intermediate of structural formula (VIb): 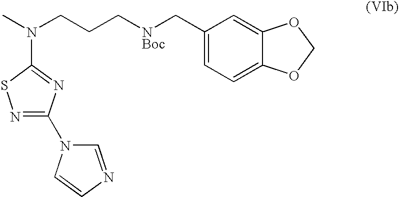
and
c) removing the Boc protecting group of structural intermediate (VIb) using trifluoroacetic acid in dichloromethane, followed by treatment with aqueous potassium carbonate solution, and isolating the desired product of structural formula (VI) in high yield and in a state of high purity.

A further embodiment of the present invention is the method for preparing the compound of structural formula (VI), wherein in step a) said trialkylamine is selected from the group consisting of triethylamine, N,N-diisopropylethylamine, 4-methylmorpholine, and N-methylpiperidine.

In other embodiments of the present invention is the method for preparing the compound of structural formula (VI), wherein in step a) triethylamine is employed as the trialkylamine.

A further embodiment of the present invention is the method for preparing the compound of structural formula (VI) wherein in step a) said suitable aprotic solvents are selected from the group consisting of dichloromethane, dimethyl sulfoxide, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H)-pyrimidinone, hexamethylphosphoramide, acetonitrile, pyridine, 2,6-lutidine, and 2,4,6-collidine; or any of these may be combined together and utilized as co-solvents.

In other embodiments of the present invention is the method for preparing the compound of structural formula (VI), wherein in step a), dichloromethane is employed as the aprotic solvent.

A further embodiment of the present invention is the method for preparing the compound of structural formula (VI), wherein in step a), a suitable temperature range is from about −70° C. to 100C.

In other embodiments of the present invention is the method for preparing the compound of structural formula (VI), wherein in step a), the suitable temperature range is from −10° C. to 40° C.

A further embodiment of the present invention is the method for preparing the compound of structural formula (VI), wherein in step a), said reaction time period ranges from about 5 minutes to 48 hours.

In other embodiments of the present invention is the method for preparing the compound of structural formula (VI), wherein in step a), said reaction time period range is from 12 to 18 hours.

A further embodiment of the present invention is the method for preparing the compound of structural formula (VI), wherein in step b), said suitable dipolar aprotic solvents are selected from the group consisting of dimethyl sulfoxide, sulfolane, N,N-dimethylformamide, N,N-dimethylacetamide, 1,3-dimethyl-3,4,5,6-tetrahydro-2(1H-pyrimidinone, and hexamethylphosphoramide; or any of these may be combined together and utilized as co-solvents.

In other embodiments of the present invention is the method for preparing the compound of structural formula (VI), wherein in step b), dimethyl sulfoxide is employed as the dipolar aprotic solvent.

A further embodiment of the present invention is the method for preparing the compound of structural formula (VI), wherein in step b), said imidazole salt derivative is selected from the group consisting of sodium, potassium, lithium and cesium.

In other embodiments of the present invention is the method for preparing the compound of structural formula (VI), wherein in step b), said imidazole salt is the sodium derivative.

A further embodiment of the present invention is the method for preparing the compound of structural formula (VI), wherein in step b), the suitable temperature range is from about −10° C. to 200° C.

In other embodiments of the present invention is the method for preparing the compound of structural formula (VI), wherein in step b), a suitable temperature range is from 10° C. to 90° C.

A further embodiment of the present invention is the method for preparing the compound of structural formula (VI), wherein in step b), a suitable time period range is from about 5 minutes to 48 hours.

In other embodiments of the present invention is the method for preparing the compound of structural formula (VI), wherein in step b), the suitable time period range is from 8 to 16 hours.

In yet further embodiments of the present invention, the method for preparing the compound of structural formula (VI): 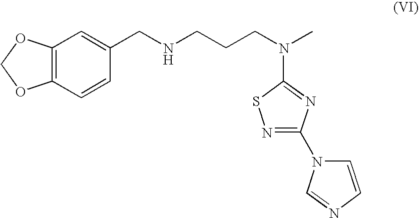
comprising:
d) reacting the compound of structural formula (III), wherein Hal=C, with benzo[1,3]dioxol-5-ylmethyl-(3-methylamino-propyl)-carbamic acid tert-butyl ester in the presence of a trialkylamine in a suitable aprotic solvent, using an appropriate reaction time over a suitable temperature range, with or without isolating the reaction product of structural formula (VIb); 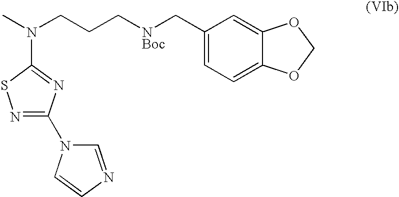
then:
e) removing the Boc protecting group of the compound of structural formula (VIb) using trifluoroacetic acid in dichloromethane, followed by treatment with aqueous potassium carbonate solution, and isolating the desired product of structural formula (VI) in high yield and in a state of high purity.

A further embodiment of the present invention is the method for preparing the compound of structural formula (VI), wherein in step d), said trialkylamine is selected from the group consisting of triethylamine, N,N-diisopropylethylamine, 4-methylmorpholine, and N-methylpiperidine.

In other embodiments of the present invention is the method for preparing the compound of structural formula (VI), wherein in step d), triethylamine is employed as the trialkylamine.

A further embodiment of the present invention is the method for preparing the compound of structural formula (VI), wherein in step d), said suitable aprotic solvents are selected from the group consisting of dichloromethane, dimethyl sulfoxide, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H)-pyrimidinone, hexamethylphosphoramide, acetonitrile, pyridine, 2,6-lutidine, and 2,4,6-collidine; or any of these may be combined together and utilized as co-solvents.

In other embodiments of the present invention is the method for preparing the compound of structural formula (VI), wherein in step d), dichloromethane is employed as the aprotic solvent.

A further embodiment of the present invention is the method for preparing the compound of structural formula (VI), wherein in step d), a suitable temperature range is from about -70° C to 100C.

In other embodiments of the present invention is the method for preparing the compound of structural formula (VI), wherein in step a), the suitable temperature range is from −10° C. to 40° C.

A further embodiment of the present invention is the method for preparing the compound of structural formula (VI), wherein in step d), said reaction time period ranges from about 5 minutes to 48 hours.

In other embodiments of the present invention is the method for preparing the compound of structural formula (VI), wherein in step d), said reaction time period range is from 12 to 18 hours.

Another embodiment of the present invention is the method for preparing the compound of structural formula (VI), 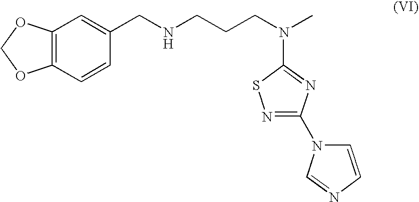
comprising:
f) Reacting the compound of structural formula (IV), wherein Hal=Cl, with benzo[1,3]dioxol-5-ylmethyl-(3-methylamino-propyl)-carbamic acid tert-butyl ester in the presence of triethylamine in dichloromethane, at about 0° C. to ambient temperature, for a time period of about 5 minutes to 48 hours, with or without isolating the reaction product of structural formula (VIa); 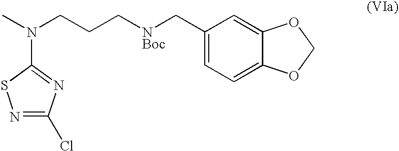
then
g) Reacting the compound of structural formula (VIa) with a salt of imidazole in a suitable dipolar aprotic solvent, at about ambient temperature to 80° C., over a time period of about 5 minutes to 48 hours, with subsequent removal of the protecting group under acidic conditions at about ambient temperature, optionally at an elevated temperature, for a time period of about 5 minutes to 24 hours, and isolating the desired product.

It should be recognized that certain modifications to the method will be obvious to those skilled in the art, and such changes are intended to be within the scope of this invention. It is intended that the method will be carried out by skilled chemists who may make changes, such as preferably, but not necessarily, carrying out sequential reactions in the same vessel, or changing solvents or reaction temperatures or equipment, especially for economic reasons. All such modifications are to be considered within the scope of this invention.

As used herein, the terms below have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon—carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The term “ambient temperature,” as used herein, alone or in combination, refers to the actual surrounding room temperature during the specified reaction period, and generally refers to a temperature range of about 20° C. to about 30° C., more preferably a temperature range of about 22° C. to about 27° C.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “ammonia equivalent,” as used herein, alone or in combination, refers to a reagent which serves as a synthetic equivalent of ammonia under the specified reaction conditions. Examples include ammonium chloride, ammonia gas, ammonium hydroxide, ammonium acetate, ammonium sulfate, ammonium bicarbonate, ammonium sulfate, and ammonium carbamate.

The term “aprotic solvent,” as used herein, alone or in combination, refers to a solvent which does not contain a hydrogen atom attached to a strongly electronegative element. It cannot donate a hydrogen atom to hydrogen bonding interactions. Examples of aprotic solvents include dichloromethane, toluene, acetone, ethyl acetate, diethyl ether, and tetrahydrofuran.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [C(O)H]and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “dipolar aprotic solvent” as used herein, alone or in combination, refers to a polar solvent possessing a comparatively high relative permittivity (or dielectric constant), greater than ca. 15, and a sizable permanent dipole moment, that cannot donate suitably labile hydrogen atoms to form strong hydrogen bonds. Examples of dipolar aprotic solvents include dimethyl sulfoxide, N,N-dimethyformamide, 1,3-dimethyl-3,4,5,6-tetrahydro-2(1H)-pyrimidinone, hexamethylphosphoramide, acetonitrile, and the like.

The term “DCM” as used herein, alone or in combination, refers to dichloromethane.

The term “DMSO” as used herein, alone or in combination, refers to dimethyl sulfoxide.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “formaldehyde equivalent,” as used herein, alone or in combination, refers to a reagent which serves as a synthetic equivalent of formaldehyde under the specified reaction conditions. Examples include formaldehyde gas, formalin, formaldehyde sodium bisulfite addition product, paraformaldehyde, methylal, and s-1,3,5-trioxane.

The term “glyoxal equivalent,” as used herein, alone or in combination, refers to a reagent which serves as a synthetic equivalent of glyoxal under the specified reaction conditions. Examples include glyoxal, glyoxal hydrate, and glyoxal bis-sodium bisulfite addition product.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The phrase “in the main chain” refers to the longest contiguous or adjacent chain of carbon atoms starting at the point of attachment of a group to the compounds of this invention.

The term “lower,” as used herein, alone or in combination, means containing from 1 to and including 6 carbon atoms.

The term “nitro,” as used herein, alone or in combination, refers to —NO₂—.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “protic solvent” as used herein, alone or in combination, refers to a solvent that carries hydrogen attached to oxygen as in a hydroxyl group or attached to nitrogen as in an amine group. Such solvents can donate an H+ (proton). Examples of protic solvents include water, ethanol, tert-butanol, and diethylamine.

The term “protic acid” refers to those acids such as HCl, H₂SO₄, H₃PO₄, p-toluenesulfonic acid, trifluoroacetic acid, acetic acid, methane sulfonic acid, or a strongly acidic cationic ion exchange resin, such as Dowex® 50 or Amberlyte® IR-112, for example.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—. The term “sulfinyl,” as used herein, alone or in combination, refers to —S(O)—.

The term “sulfonyl,” as used herein, alone or in combination, refers to —S(O)₂.

The term “N-sulfonamido” refers to a RS(═O)₂NR′-group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′—group, with R and R′ as defined herein.

The term “O-thiocarbamyl” refers to a —OC(S)NRR′, group with R and R′ as defined herein.

The term “TBME” refers to tert-butyl methyl ether.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

When a particular measure or amount is referred to, unless otherwise stated, said amount should be understood to be reasonably approximate, “reasonable” being subject to interpretation by one of skill in the art. Those of skill in the art regularly employ minor variations in quantitation to achieve ideal results. Furthermore, weights and measures are subject to variation based upon the sensitivity of the apparatus.

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

Thus, preferred salts include acetate, hydrochloride, adipate, benzoate, fumarate, gentisic acid, glutarate, 1-hydroxy-2-naphthoate, maleate, L-malate, malonate, succinate , L-(+)-tartrate acid, citrate, glycolate, D,L-tartrate, phosphate, p-toluenesulfonate, methanesulfonate, nicotinate, and p-hydroxybenzoate salts of compounds of the present invention. A salt of a compound can be made by reacting the appropriate compound in the form of the free base with the appropriate acid.

### General Synthetic Methods for Preparing Compounds

The following schemes and examples can be used to practice the present invention. Starting materials are commercially available, made by known procedures, or prepared as illustrated herein.

One of the principal routes for preparation of compounds within the scope of the instant invention is depicted in Scheme 1. According to this route, the 3-amino-5-halo-[1,2,4]thiadiazole derivative (102) is reacted with a mixture of glyoxal, formalin, and ammonium chloride in the presence of a protic acid catalyst such as phosphoric acid, in a protic solvent such as ethanol and water, to afford the 5-halo-3-imidazol-1-yl-[1,2,4]thiadiazole intermediate (103). Optional protic solvents, which may also be used as co-solvents for this reaction step, include methanol, propanol, iso-propanol, butanol, tert-butanol, methoxyethanol, ethoxyethanol, ethylene glycol, propylene glycol and the like. Temperature range for this reaction step is from about −20° C. to 150° C., the preferred range being from about 40° C. to 90° C. Required time for this reaction step ranges from 5 minutes to 48 hours, the preferred range being about 16 to 20 hours. Intermediate (103) is then reacted with an amine derivative of formula R¹⁰¹N(H)R¹⁰² in a aprotic solvent such as dichloromethane to afford the 3-imidazol-1-yl-[1,2,4]thiadiazol-5-yl)-dialkyl-amine derivative (100). Optional aprotic solvents, which may also be used as co-solvents for this reaction step, include DMSO, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H)-pyrimidinone, hexamethylphosphoramide, acetonitrile, pyridine, 2,6-lutidine, 2,4,6-collidine, and the like. Temperature range for this reaction step is from about −70° C. to 100° C., the preferred range being from about −10° C. to 40° C. Required time for this reaction step ranges from 5 minutes to 48 hours, the preferred range being about 16 to 20 hours.

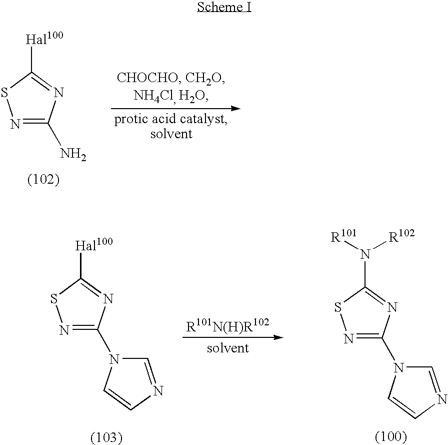

Another principal route for preparation of compounds within the scope of the instant invention is depicted in Scheme 2. According to this route, the 3,5-dihalo-[1,2,4]thiadiazole derivative (104) is reacted with an amine derivative of formula R¹⁰¹N(H)R¹⁰² in a aprotic solvent such as dichloromethane to afford the 3-halo-N,N-dialkyl-1,2,4-thiadiazol-5-amine derivative (105). Optional aprotic solvents, which may also be used as co-solvents for this reaction step, include DMSO, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H)-pyrimidinone, hexamethylphosphoramide, acetonitrile, pyridine, 2,6-lutidine, 2,4,6-collidine, and the like. Temperature range for this reaction step is from about −70° C. to 100° C., the preferred range being from about −10° C. to 40° C. Required time for this reaction step ranges from 5 minutes to 48 hours, the preferred range being about 15 to 24 hours. Intermediate (105) is then reacted with reacted with the sodium, potassium, lithium, or cesium salt of an optionally substituted imidazole or 1,2,4-triazole derivative in a dipolar aprotic solvent, such as DMSO, to afford the 3-imidazol-1-yl-[1,2,4]thiadiazol-5-yl)-dialkyl-amine derivative (100). The sodium imidazole or 1,2,4-triazole salt derivative is preferred. Optional dipolar aprotic solvents, which may also be used as co-solvents for this reaction step, include DMSO, sulfolane, N,N-dimethylformamide, N,N-dimethylacetamide, 1,3-dimethyl-3,4,5,6-tetrahydro-2(1H)-pyrimidinone, hexamethylphosphoramide, and the like. Temperature range for this reaction step is from about −10° C. to 200° C., the preferred range being from about 0° C. to 90° C. Required time for this reaction step ranges from 5 minutes to 48 hours, the preferred range being about 8 to 16 hours.

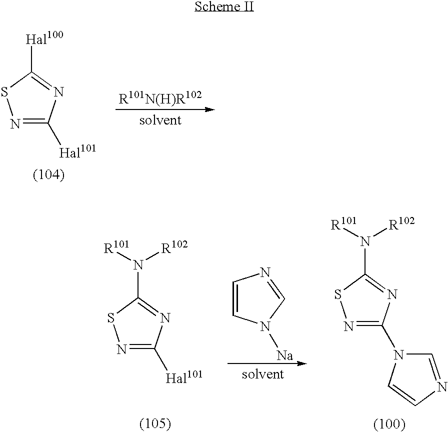

Examples 1-64 can be synthesized using the general synthetic procedures set forth in Scheme 1 and Scheme 2. In some cases the order of carrying out the foregoing reaction scheme may be varied to facilitate the reaction or to avoid unwanted reaction products. The following examples are provided for the purpose of further illustration only and are not intended to be limitations on the disclosed invention.

The invention is further illustrated by the following examples.

### EXAMPLE 1

### N′-Benzo[1,3]-dioxol-5-ylmethyl-N-(3-imidazol-1-yl-[1,2,4]thiadiazol-5-yl)-N-methyl-propane-1,3-diamine

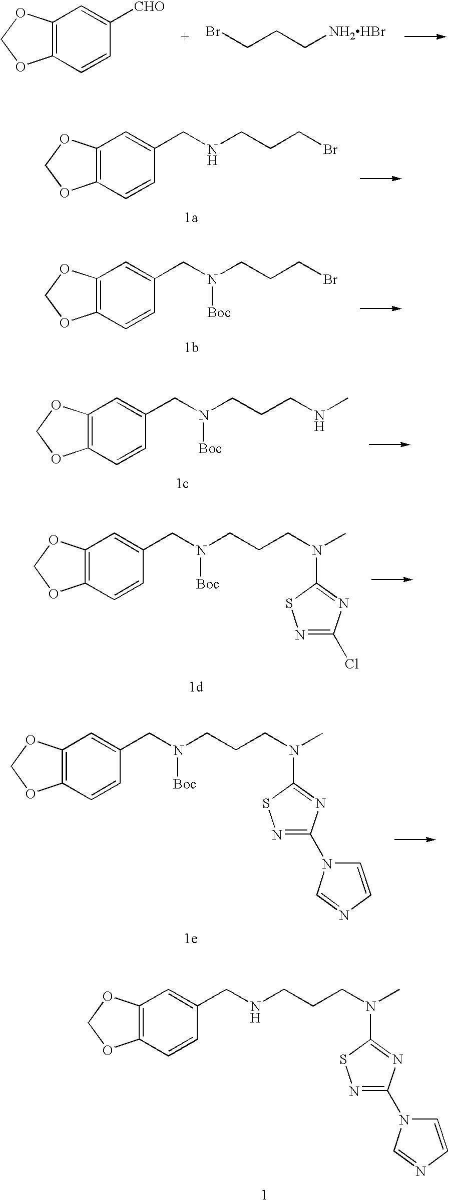 Step 1:
Preparation of example 1a: Benzo[1,3]dioxo-5-ylmethy-(3-bromo-propyl)-amine

Triethylamine (1.30 L, 9.30 mol) was added to a suspension of 3-bromopropan-1-amine hydrobromide (2.00 kg, 9.10 mol) in CH₂Cl₂ (16.0 L) at 22° C. under nitrogen. The solution was stirred for 15 minutes prior to the addition of piperonal (1.30 kg, 8.70 mol). The mixture was heated to 40° C. for 2.5 h and cooled to room temperature. Water (9.00 L) was added to the suspension and the mixture was stirred for 20 minutes. The layers were separated and organic layer was concentrated under vacuum to a yellow oil Isopropanol (16.0 L) and acetic acid (1.50 L) were added to the oil The solution was cooled to 15° C. under nitrogen and sodium triacetoxyborohydride (2.20 kg, 10.4 mol) was added in 50 g portions over 1 h. The mixture was stirred at room temperature for 14 h prior to cooling to 15° C. Water (6 L) was added while maintaining an internal temperature below 26° C. The pH was adjusted to 7-8 with the sat. aqueous K₂CO₃ followed by the addition of brine (10.0 L). The precipitate was collected by vacuum filtration and washed with water (10.0 L) . The solid was dried overnight under vacuum to afford 1.24 kg (53%/) of benzo[1,3]dioxol-5-ylmethyl-(3-bromo-propyl)-amine (la) as a white solid. [M+H]⁻271.90, 273.94; ¹H-NMR (400 MHz, DMSO) δ 7.25 (s, 1H), 7.04 (d, 1H), 6.96 (d, 1H), 6.05 (s, 2H), 4.04 (s, 2H), 3.61 (t, 2H), 2.94 (t, 2H), 2.24 (t, 2H); ¹³C-NMR (100 MHz, DMSO) δ 148.1, 147.7, 126.1, 124.6, 110.8, 108.7, 101.8, 50.1, 45.2, 31.9, 29.1

Step 2:

Preparation of Example 1b: Benzo[1,3]dioxol-5-ylmethyl-(3-bromo-propyl)-carbamic Acid tert-butyl Ester

Triethylamine (1.24 L, 8.90 mol) was added over 45 minutes to a mixture of benzo[1,3]dioxol-5-ylmethyl-(3-bromo-propyl)-amine (2.20 kg, 8.10 mol) and di-tert-butyl dicarbonate (1.94 kg, 8.90 mol) in MeOH (20.0 L) at 20-24° C. under nitrogen. The solution was stirred for 1h at room temperature. The mixture was concentrated under vacuum (70-15 torr) at 32° C. prior to the addition of ethyl acetate (5.00 L) and water (3.00 L). The layers were separated and the aqueous back extracted with ethyl acetate (1.00 L). The combined organic layers were concentrated under vacuum (70-15 torr) at 32° C. to give 2.93 kg (97%) of benzo[1,3]dioxol-5-ylmethyl-(3-bromo-propyl)-carbamic acid tert-butyl ester (1b) as an amber oil.

Step 3:

Preparation of Example 1c: Benzo[1,3]dioxol-5-ylmethyl-(3-methylamino-propyl)-carbamic Acid tert-butyl Ester

Methylamine (33 wt. % in EtOH, 30.0 L, 240 mol) was added over 3 h to a solution of benzo[1,3]dioxol-5-ylmethyl-(3-bromo-propyl)-carbamic acid tert-butyl ester (2.93 kg, 7.90 mol) in EtOH (4.00 L) while maintaining an internal temperature of 14-17° C. The reaction mixture was warmed to room temperature and stirred for 14 h. The solution was concentrated under vacuum (70-15 torr) at 32° C. then partitioned between ethyl acetate (5.00 L) and water (3.00 L). The phases were separated and the aqueous layer back extracted with ethyl acetate (2.00 L). The combined organic layers were concentrated under vacuum (70-5 torr) at 32° C. to give 2.59 kg (100%) of benzo[1,3]dioxol-5-ylmethyl-(3-methylamino-propyl)-carbamic acid tert-butyl ester (1c) as a clear oil. [M+H]⁺323.70.

Step 4:

Preparation of Example 1d: Benzo[1,3]dioxol-5-ylmethyl-{3-[(3-chloro-[1,2,4]thiadiazol-5-yl)-methyl-amino]-propyl}-carbamic Acid tert-butyl Ester

A solution of benzo[1,3]dioxol-5-ylmethyl-(3-methylamino-propyl)-carbamic acid tert-butyl ester (2.59 kg, 7.90 mol) in CH₂Cl₂ (20.0 L) was cooled to 7.5° C. under nitrogen. Triethylamine (2.20 L, 15.8 mol) was added and the solution was cooled to 0.5° C. 3,5-Dichloro-1,2,4-thiadiazole (1.22 kg, 7.90 mol) was added over 2 h while maintaining an internal temperature of 0-2° C. The reaction mixture was warmed room temperature and stirred for 15 h. Water (9.00 L) was added and the organic layer was separated. The solution was concentrated under vacuum (220-10 torr) at 32° C. to give 3.26 kg (94%) of benzo[1,3]dioxol-5-ylmethyl-{3-[(3-chloro-[1,2,4]thiadiazol-5-yl)-methyl-amino]-propyl}-carbamic acid tert-butyl ester (1d) as an amber oil. [M+H]⁺441.37; ¹H-NMR (400 MHz, CD₃OD) δ 6.77 (m, 3H), 5.96 (s, 2H), 4.35 (s, 2H), 3.4-3.0 (m, 6H), 1.84 (br s, 3H), 1.50 (s, 9H).

Step 5:

Preparation of Example 1e: Benzo[1,3]dioxol-5-ylmethyl{-3-[(3-imidazol-t-yl-[1,2,4]thiadiazol-5-yl)-methyl-amino]-propyl}-carbamic Acid tert-butyl Ester

Sodium imidazole (2.10 kg, 23.1 mol) was added to a solution of benzo[1,3]dioxol-5-ylmethyl-{3-[(3-chloro-[1,2,4]thiadiazol-5-yl)-methyl-amino]-propyl}-carbamic acid tert-butyl ester (3.00 kg, 6.80 mol) in DMSO (8.00 L) at 22° C. under nitrogen. The solution was heated at 74° C. for 13 h then cooled to room temperature and stirred for 7 h. Citric acid (10 L of a 5% aqueous solution) was added over 8 hours and the solution was extracted with ethyl acetate (10.0 L). The layers were separated and the organic layer was concentrated to give 3.18 kg (99%) of benzo[1,3]dioxol-5-ylmethyl-{3-[(3-imidazol-1-yl-[1,2,4]thiadiazol-5-yl)-methyl-amino]-propyl}-carbamic acid tert-butyl ester (1e) as a green oil. [M+H]⁺473.06; ¹H-NMR (400 MHz, CD₃OD) δ 8.32 (s, 1H), 7.68 (s, 1H), 7.12 (s, 1H), 6.62-6.80 (m, 3H), 5.96 (s, 2H), 4.38 (s, 2H), 3.0-3.6 (m, 6H), 1.88 (br s, 3H), 1.52 (s, 9H).

Step 6:

Preparation of Example 1: N′-Benzo[1,3]dioxol-5-ylmethyl-N-(3-imidazol-1-yl-[1,2,4]thiadiazo-5-yl)-N-methyl-propane-1,3-diamine

A solution of benzo[1,3]dioxol-5-ylmethyl-{3-[(3-imidazol-1-yl-[1,2,4]thiadiazol-5-yl)-methyl-amino]-propyl}-carbamic acid tert-butyl ester (10.6 g, 22.4 mmol) in a mixture of TFA/DCM (70 mL of a 1:1 mixture) was stirred at room temperature for 30 min. The solution was concentrated under vacuum and a saturated aqueous solution of K₂CO₃ (50 mL) was added. The mixture was extracted with ethyl acetate (2×200 mL) and the combined organics were concentrated under vacuum to give 8.30 g (99%) of N-benzo[1,3]dioxol-5-ylmethyl-N-(3-imidazol-1-yl-[1,2,4]thiadiazol-5-yl)-N-methyl-propane-1,3-diamine (1) as a colorless oil. [M+H]⁺373.26; ¹H-NMR (400 MHz, CD₃OD) δ 8.28 (s, 1H), 7.63 (s, 1H), 7.07 (s, 1H), 6.79 (s, 1H), 6.72 (s, 2H), 5.92 (s, 2H), 3.67 (s, 3 H), 3.60 (br s, 1H), 3.10 (br s, 2H), 2.66 (t, 2H), 2.0 (br s, 2H), 1.87 (q, 2 H).

### EXAMPLE 2

### N′-Benzo[1,3]dioxol-5-ylmethyl-N-(3-imidazol-1-yl-[1,2,4]thiadiazol-5-yl)-N-methyl-propane-1,3-diamine Hydrochloride Salt

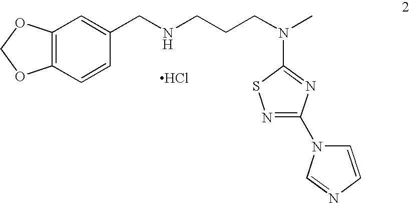

A suspension of 1 (11.4 g, 30.6 mmol) in EtOH (60 mL) was heated to 55° C. for 15 minutes to afford a clear solution. Concentrated HCl (2.63 mL, 31.5 mmol) was added causing immediate precipitation. The suspension was stirred for an additional 15 minutes at 55° C. then n-heptane (110 mL) was added and the mixture was cooled to room temperature. The precipitate was collected by vacuum filtration and washed with n-heptanes (30 mL) to afford 11.19 g (90%) of 2 as a white solid. [M+H]⁺373.13; ¹H-NMR (400 MHz, DMSO) δ 9.59 (s, 2H), 8.14 (s, 1H), 7.67 (s, 1H), 7.20 (s, 1H), 6.98 (d, 1H), 6.87 (d, 1H), 6.01 (s, 2H), 4.00 (t, 2 H), 3.82-3.68 (br s, 2H), 3.20-3.00 (br s, 3H), 2.86 (m, 2H), 2.09 (quint, 2H); Elemental found (calc) C 49.70 (49.93), H 5.17 (5.18), N 20.36 (20.55), S 7.78 (7.84), C18.89 (8.67).

### EXAMPLE 3

### N′-Benzo[1,3]dioxol-5-ylmethyl-N-(3-imidazol-1-yl-[1,2,4]thiadiazol-5-yl)-N-methyl-propane-1,3-diamine Acetate Salt

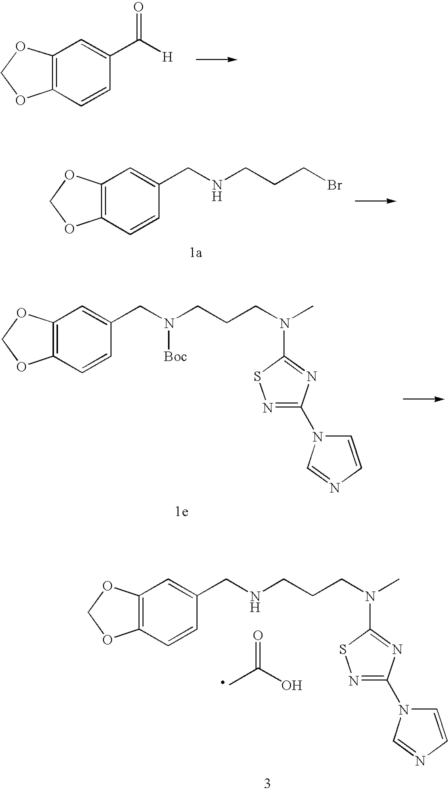 Step 1:
Preparation of Example 1a: Benzo[1,3]dioxol-5-ylmethyl-(3-bromo-propyl)-amine

Isopropanol (24.0 L) was added to a nitrogen purged reactor charged with piperonal (3.018 kg, 20.12 mol) and 3-bromopropan-1-amine hydrobromide (4.3995 kg, 20.10 mol). The resulting suspension was stirred until complete dissolution was observed (30 minutes) prior to the addition of triethylamine (2.0357 kg, 20.12 mol) via a feeding vessel. The feeding vessel was rinsed with isopropanol (0.800 L) and added to the reaction mixture. The mixture was stirred at 20° C. for 43 minutes and the resulting suspension was filtered. The vessel and filtered cake were washed with isopropanol (2×7.500 L) and combined with the mother liquor. The solution was transferred to a reactor and cooled to 5° C. prior to the addition of acetic acid (3.622 kg, 60.34 mol). NaHB(OAc)₃ (5.3670 kg, 25.32 mol) was added in ten portions over 51 minutes via a Müller barrel while maintaining an internal temperature of 5.2-9.6° C. The mixture was warmed to 22.0° C., stirred for 35 minutes then cooled to 14.6° C. Water (75.0 L) was slowly added to the mixture while maintaining an internal temperature of 14.6-21.1° C. The pH of the solution was adjusted to 7-8 with the addition of K₂CO₃ (18.0 L of a 19.4% aqueous solution) at an internal temperature of 21.1° C. Sodium chloride (37.0 L of a 23.1% aqueous solution) was added causing mass precipitation. The mixture was stirred for 30 minutes before filtration of the precipitate. The vessel and the filter cake were rinsed with water (2×30.0 L). The filter cake was dried under nitrogen and transferred into a tarred flask. The solid was dried for 44.25 hours, using a rotary evaporator, at a bath temperature of 40° C. and a pressure of 8 mbar, to give 3.778 kg (69%) of benzo[1,3]dioxol-5-ylmethyl-(3-bromo-propyl)-amine (la) as an off-white solid. [M+H]⁺271.90, 273.94; ¹H-NMR (400 MHz, DMSO) δ 7.25 (s, 1H), 7.04 (d, 1H), 6.96 (d, 1H), 6.05 (s, 2H), 4.04 (s, 2H), 3.61 (t, 2H), 2.94 (t, 2H), 2.24 (t, 2H); ¹³C-NMR (100 MHz, DMSO) δ 148.1, 147.7, 126.1, 124.6, 110.8, 108.7, 101.8, 50.1, 45.2, 31.9, 29.1

Step 2:

Preparation of Example 1e: Benzo[1,3]dioxol-5-ylmethyl-{3-[(3-imidazol-1-yl-[1,2,4]thiadiazol-5-yl)-methyl-amino]-propyl}-carbamic Acid tert-butyl Ester

1a (4.05 kg, 14.9 mol) and Boc₂O (3.26 kg, 14.9 mol) were added to a nitrogen purged 160 L reactor followed by the addition of methanol (45.0 L) via a feeding vessel. A solution of triethylamine (1.51 kg, 14.9 mol) and methanol (11.0 L) was added over a period of 21 min to the reaction mixture, and the resulting solution was maintained at an internal temperature of 20-21° C. for 45 min. The reaction mixture was transferred to the feeding vessel and the reactor was washed with methanol (11.0 L) and combined with the reaction mixture. The reactor, equipped with a 6N sulfuric acid filled scrubber, was charged with a solution of methylamine in ethanol (8N, 55.5L, 444 mol) and the reaction mixture was slowly added from the feeding vessel over 2.1 h while maintaining an internal temperature of 20-21° C. The solution was maintained at an internal temperature of 20° C. for 37.5 h before removal of 45.0 L of solvent by vacuum distillation, using an external vacuum pump connected to the scrubber, at a pressure ranging from 271 to 45 mbar and a jacket temperature of 49° C., to afford an oil. DCM (16.0 L) and an aqueous solution of Na₂CO₃ (9.5%, 32.4 L) were added to the oil and stirred at 19-21° C. for 13 minutes. The separated aqueous layer was back extracted with DCM (16.0 L) and the combined organic layers were washed with water (16.0 L). The separated organics were concentrated through azeotropic distillation, at an internal temperature of 22-23° C. and a pressure of 503-501 mbar, to yield a pale brown solution. The solution and TEA (4.74 kg, 46.8 mol) were charged into a nitrogen purged 160 L reactor. A solution of 3,5-dichloro-1,2,4-thiadiazole (2.49 kg, 16.1 mol) in DCM (20.0 L) was added to the reaction mixture from the feeding vessel, over 48 min, while maintaining an internal temperature of 18-22° C. The reaction mixture was maintained at 18-20° C. for 16.4 hours followed by addition of water (40 L) and the resulting mixture was stirred at an internal temperature of 20° C. for 7 min. To the separated organic layer was added an aqueous solution of NaCl (half saturated, 20 L). The resulting mixture was stirred for 6 min at an internal temperature of 20° C. before transferring the organic layer into the reaction vessel and removing 55 L of solvent by distillation at an internal temperature of 19-28° C. and a pressure of 500-300 mbar. Residual DCM was removed by iterative distillation with TBME (3×41 L) at an internal temperature of 14-27° C. and a pressure of 244-75 mbar. DMSO (35 L) was added and the vacuum was released, yielding a solution. Sodium imidazole (4.22 kg, 46.9 mol) was added and the resulting mixture was heated to an internal temperature of 80° C. over 2.13 hours and maintained at 80° C. for an additional 9.85 h. The reaction was then cooled to 20° C. followed by addition of water (35 L) over 1 h at an internal temperature of 20-23° C. ⁱPrOAc (35 L) was added and the mixture was stirred for 6 minutes. The separated aqueous layer was extracted with ⁱPrOAc (17 L) and the combined organic layers were washed sequentially with brine (34 L), citric acid (34 L of a 5% aqueous solution) and brine (18 L). The organic layer was concentrated to an oil by distillation at an internal temperature of 19-27° C. and a pressure of 195-64 mbar. The oil was dried for 71.5 hours at an external temperature of 20-40° C. and a pressure of 53-8 mbar prior to manual removal of paraffin oil (0.341 kg) to give 6.55 kg (93%) of benzo[1,3]dioxol-5-ylmethyl-{3-[(3-imidazol-1-yl-[1,2,4]thiadiazol-5-yl)-methyl-amino]-propyl}-carbamic acid tert-butyl ester (1e) as a pale brown oil. [M+H]⁺473.06; ¹H-NMR (400 MHz, CD₃OD) 6 8.32 (s, 1H), 7.68 (s, 1H), 7.12 (s, 1H), 6.62-6.80 (m, 3H), 5.96 (s, 2H), 4.38 (s, 2H), 3.0-3.6 (m, 6H), 1.88 (br s, 3H), 1.52 (s, 9H).

Step 3:

Preparation of Example 3: N′-Benzo[1,3]dioxol-5-ylmethyl-N-(3-imidazol-1-yl-[1,2,4]thiadiazol-5-yl)-N-methyl-propane-1,3-diamine Acetate Salt

1e (6.69 kg, 14.2 mol) was dissolved in isopropanol (1.34 L) and TBME (5.5 L). The resulting solution was added to a nitrogen purged 160 L reactor, equipped with a scrubber filled with water (40.0 L), and the feeding vessel was rinsed with TBME (48.0 L). The rinsing solvent was added to the reactor and the solution was heated to 35° C. over 22 min. A solution of HCl (8.07 kg, 221 mol) in water (2.0 L) was added to the reaction mixture over 32 min at an internal temperature of 34-37° C. The reaction mixture was maintained for 1 h at 34-37° C. with subsequent cooling to 19° C. The organic layer was discarded and the aqueous layer was treated with methanol (8.0 L) and TBME (72.0 L). An aqueous solution of K₂CO₃ (25%, 53.5 L) was added over 20 min at an internal temperature of 20-23° C. and the mixture was stirred for 1 h at 20-23° C. The layers were separated and the aqueous layer was back extracted with a mixture of methanol (2.8 L) and TBME (24.0 L). The combined organic layers were added to solid Na₂CO₃ (0.838 kg, 9.97 mol) and stirred for 12 minutes. The resulting suspension was filtered and the filter cake was washed with TBME (6.0 L). The filtrate was transferred to the reactor and 99.0 L of solvent was removed by distillation at an internal temperature of 20-36° C. and a pressure of 304-203 mbar. Isopropanol (27.0 L) was added and 27.5 L of solvent was removed by distillation at an internal temperature of 32-40° C. and a pressure of 94-44 mbar. Additional isopropanol (25.5 L) was added and the solution was filtered twice through an inline filter and heated to 55° C. Sequential addition, through inline filtration, of acetic acid (0.871 kg, 14.5 mol) and isopropanol (0.350 L) afforded a suspension that was stirred for 30 min at an internal temperature of 55° C. prior to addition of heptanes (51.0 L), through an inline filter, at an internal temperature of 51-56° C. The reaction mixture was slowly cooled to 20° C. over 4.5 h and maintained at an internal temperature of 20° C. for 9.67 hours. The resulting suspension was filtered, the reactor and filter cake were rinsed with inline filtered heptanes (2×16.0 L) and the filter cake was dried with a stream of nitrogen for 3 h. The solid was dried at an external temperature of 35-45° C. and a pressure of 53-8 mbar for 20 hours, affording 4.38 kg (72%) of 3 as a white to off-white solid. [M+H]⁺373.40; ^{H-NMR (}400 MHz, DMSO) δ 8.28 (s, 1H), 7.70 (s, 1H), 7.06 (s, 1H), 6.90 (d, 1H), 6.80 (d, 1H), 6.76 (d, 1H), 5.95 (s, 2H), 3.65 (s, 2H), 3.70-3.54 (br s, 1H), 3.20-3.04 (br s, 4H), 2.56 (t, 2H), 2.47 (m, 2H), 1.89 (s, 3H), 1.81 (quint, 2H); Elemental found (calc) C 52.80 (52.76), H 5.58 (5.59), N 19.40 (19.43), S 7.37 (7.41).

### EXAMPLE 4

### N′-Benzo[1,3]dioxol-5-ylmethyl-N-(3-imidazol-1-yl-[1,2,4]thiadiazol-5-yl)-N-methyl-propane-1,3-diamine Adipate Salt

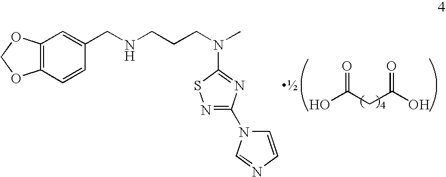

A suspension of 1 (15.3 g, 41.08 mmol) in EtOH (82 mL) was heated to 55° C. for 15 minutes to afford a clear solution. Adipic acid (3.06 g, 20.95 mmol) was added causing immediate precipitation. The suspension was stirred for an additional 15 minutes at 55° C. then n-heptane (164 mL) was added and the mixture was cooled to rt. The solid was collected by vacuum filtration and washed with n-heptanes (200 mL) to afford 15.62 g (85%) of 4 as a white solid. [M+H]⁺373.23; ¹H-NMR (400 MHz, CD₃OD) δ 8.36 (t, 1H), 7.75 (t, 1H), 7.08 (t, 1H), 6.88 (d, 1H), 6.83 (dd, 1H), 6.75 (d, 1H), 5.94 (s, 2H), 3.93 (s, 2H), 3.80-3.64 (br s, 2H), 3.14 (br s, 3H), 2.90 (m, 2H), 2.22 (m, 2H), 2.05 (quint, 2H), 1.62 (m, 2H); Elemental found (calc) C 53.73 (53.92), H 5.61 (5.66), N 18.62 (18.86), S 7.11 (7.20).

### EXAMPLE 5

### N-1-(3-Imidazol-1-yl-[1,2,4]thiadiazol-5-yl)-N-1-methyl-propane-1,3-diamine

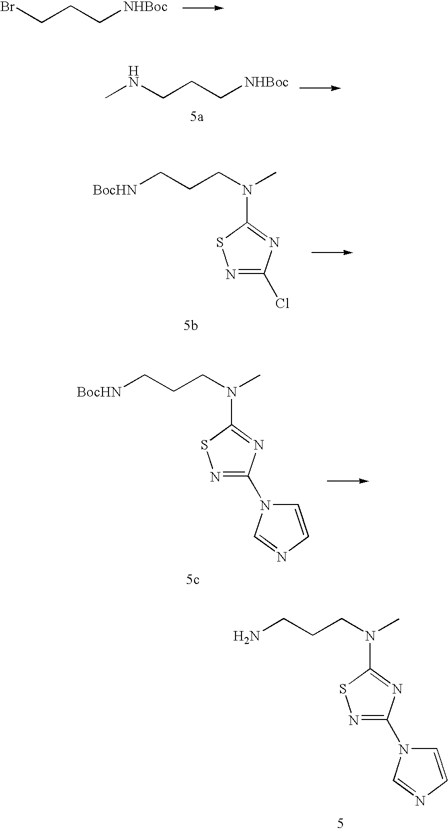 Step 1:
Preparation of Example 5a: (3-Methylamino-propyl)-carbamic Acid tert-butyl Ester

Methylamine (100 mL of a 2.00 M solution in THF, 200 mmol) was added to (3-bromo-propyl)-carbamic acid tert-butyl ester (11.2 g, 47.0 mmol) at room temperature under nitrogen and the solution was stirred for 4 h. The resulting suspension was filtered and concentrated under reduced pressure to give 7.58 g (86%) of (3-methylamino-propyl)-carbamic acid tert-butyl ester (5a) as a clear oil. [M+H]⁺188.94.

Step 2:

Preparation of Example 5b: {3-[(3-Chloro-[1,2,4]thiadiazol-5-yl)-methyl-amino]-propyl}-carbamic Acid tert-butyl Ester

Triethylamine (3 mL) was added to a solution of (3-methylamino-propyl)-carbamic acid tert-butyl ester (7.00 g, 36.8 mmol) and 3,5-dichloro-[1,2,4]thiadiazole (4.75 g, 30.7 mmol) in DMSO (150 mL) at room temperature under nitrogen. The mixture was stirred for 24 h prior to the addition of brine (100 mL). The solution was extracted with DCM (3×300 mL) and the combined organic layers were concentrated under vacuum. The product was purified using column chromatography (DCM to 9:1 DCM/MeOH) to give 6.5 g (69%) of {3-[(3-chloro-[1,2,4]thiadiazol-5-yl)-methyl-amino]-propyl}-carbamic acid tert-butyl ester (5b) as a clear oil. [M+H]⁺307.40.

Step 3:

Preparation of Example 5c: {3-[(3-Imidazol-1-yl-[1,2,4]thiadiazol-5-yl)-methyl-amino]-propyl}-carbamic Acid tert-butyl Ester

Sodium hydride (833 mg of a 60% dispersion on mineral oil, 57.8 mmol) was added to a solution of {3-[(3-chloro-[1,2,4]thiadiazol-5-yl)-methyl-amino]-propyl}-carbamic acid tert-butyl ester (6.50 g, 21.2 mmol) and imidazole (7.20 g, 105 mmol) in DMSO (100 mL) at room temperature under nitrogen. The reaction mixture was heated to 60° C. and stirred for 14 h. The mixture was cooled to room temperature prior to the addition of brine (400 mL). The solution was extracted with DCM (2×150 mL) and the combined organic layers were concentrated. The product was purified by column chromatography (Hex to 1:4 Hex/EtOAc) to give 6.78 g (94%) of {3-[(3-imidazol-1-yl-[1,2,4]thiadiazol-5-yl)-methyl-amino]-propyl}-carbamic acid tert-butyl ester (5c) as a clear oil. [M+H]⁺339.10.

Step 4:

Preparation of Example 5: N-1-(3-Imidazol-1-yl-[1,2,4]thiadiazol-5-yl)-N-1-methyl-propane-1,3-diamine was prepared following the procedures described in preparation of Example 1. [M+H]⁺239.08.

### EXAMPLE 6

### N′-(4-Chloro-benzyl)-N-(3-imidazol-1-yl-[1,2,4]thiadiazol-5-yl)-N-methyl-propane-1,3-diamine

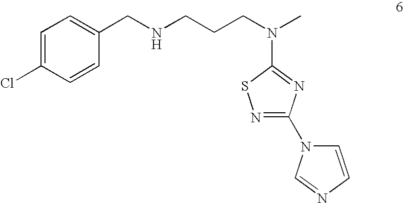

A mixture of N-1-(3-Imidazol-1-yl-[1,2,4]thiadiazol-5-yl)-N-1-methyl-propane-1,3-diamine (5) (150 mg, 0.64 mmol), 4-chloro-benzaldehyde (90 mg, 0.64 mmol), EtOH (2.0 mL) and glacial acetic acid (150 μL) was heated to 60° C. and stirred for 14 h. The solution was concentrated under vacuum prior to the addition of EtOH (2.0 mL). The mixture was cooled to 0° C. and sodium triacetoxyborohydride (270 mg, 1.3 mmol) was added. The reaction was vented and stirred at room temperature for 22 h. The resulting suspension was concentrated under vacuum to afford a residue. DCM (25 mL) was added and the solution was washed with NaHCO₃ (2×25 mL of a sat. aq. solution). The organic layer was concentrated under vacuum and the product was purified by flash silica chromatography (DCM to 1:19 MeOH/DCM) to give 37 mg (16%) of 6 as a clear oil. [M+H]⁺363.00; ¹H NMR (400 MHz, CDCl₃) δ 8.30 (d, 1H), 7.64 (s, 1H), 7.25 (q, 4H), 7.09 (d, 1H) 3.75 (s, 2H), 3.62 (t, 2H), 3.12 (s, 3H), 2.68 (t, 2H), 1.86 (q, 2H), 1.78 (s, 1H).

### EXAMPLE 7

### N-(3-Imidazol-1-yl-[1,2,4]thiadiazol-5-yl)-N′-(4-methoxy-benzyl)-N-methyl-propane-1,3-diamine

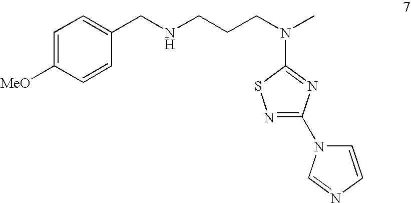 Example 7 was prepared following the procedures described in preparation of Example 6 using 4-methoxybenzaldehyde. [M+H]⁺360.40; ¹H NMR (400 MHz, CDCl₃) δ 8.35 (s, 1H), 7.67 (s, 1H), 7.25 (d, 2H), 7.13 (s, 1H) 6.86 (d, 2H), 3.82 (s, 3H), 3.79 (s, 2H), 3.52 (d, 2H), 3.12 (s, 1H), 3.12-2.73 (m, 2H), 2.07 (s, 3H), 2.07-1.96 (m, 2H).

### EXAMPLE 8

### N′-(3,4-Difluoro-benzyl)-N-(3-imidazol-1-yl-[1,2,4]thiadiazol-5-yl)-N-methyl-propane-1,3-diamine

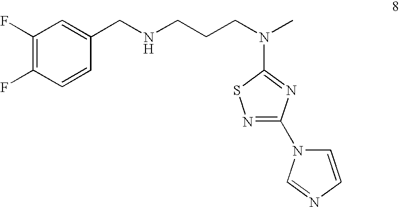

Example 8 was prepared following the procedures described in preparation of Example 6 using 3,4-difluoro-benzaldehyde. [M+H]⁺365.01; ¹H NMR (400 MHz, CDCl₃) δ 8.31 (s, 1H), 7.65 (d, 1H), 7.18-7.01 (m, 4H), 3.74 (s, 2H) 3.70-3.49 (m, 2H), 3.14 (s, 3H), 2.70-2.67 (t, 2H), 1.93-1.87 (m, 2H)

### EXAMPLE 9

### N′-(2,6-Dichloro-benzyl)-N-(3-imidazol-1-yl-[1,2,4]thiadiazol-5-yl)-N-methyl-propane-1,3-diamine

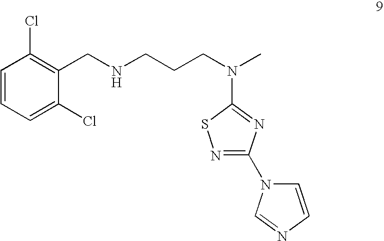 Example 9 was prepared following the procedures described in preparation of Example 6 using 2,6-dichloro-benzaldehyde. [M+H]⁺398.90; ¹H NMR (400 MHz, CDCl₃) δ 9.31 (s, 1H), 7.93 (s, 1H), 7.44 (s, 1H), 7.36-7.28 (m, 3H) 4.15 (s, 2H), 3.79 (s, 1H), 3.22-3.18 (t, 2H), 3.12 (s, 2H), 2.68 (s, 3H), 2.30-2.27 (m, 2H)

### EXAMPLE 10

### N-(3-Imidazol-1-yl-[1,2,4]thiadiazol-5-yl)-N-methyl-N′-(4-trifluoromethyl-benzyl)-propane-1,3-diamine

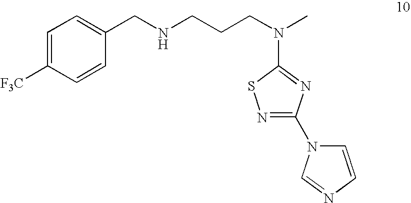 Example 10 was prepared following the procedures described in preparation of Example 6 using 4-trifluoromethyl-benzaldehyde. [M+H]⁺396.70; ¹H NMR (400 MHz, CDCl₃) δ 9.49 (s, 1H), 7.93 (s, 1H), 7.60 (s, 4H), 7.39 (s, 1H) 4.23 (s, 2H), 3.80 (br s, 1H), 3.51 (s, 2H), 3.12-3.09 (m, 2H), 2.68 (s, 3H), 2.20-2.14 (m, 2H)

### EXAMPLE 11

### N′-Benzyl-N-(3-imidazol-1-yl-[1,2,4]thiadiazol-5-yl)-N-methyl-propane-1,3-diamine

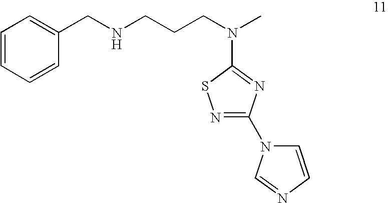 Example 11 was prepared following the procedures described in preparation of Example 6 using benzaldehyde. [M+H]⁺329.20; ¹H NMR (400 MHz, CDCl₃) δ 8.32 (s, 1H), 7.67 (s, 1H), 7.35-7.28 (m, 5H), 7.12-7.11 (d, 1H), 3.80 (s, 2H), 3.52 (m, 2H), 3.15 (s, 3H), 2.74-2.71 (t, 2H), 1.93-1.90 (m, 2H), 1.66 (s, 1H).

### EXAMPLE 12

### N′-(2,3-Dihydro-benzo[1,4]dioxin-6-ylmethyl)-N-(3-imidazol-1-yl-[1,2,4]thiadiazol-5-yl)-N-methyl-propane-1,3-diamine

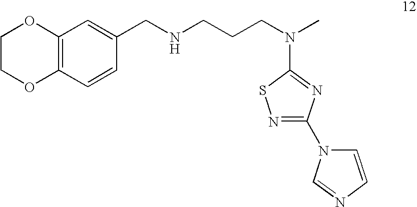 Example 12 was prepared following the procedures described in preparation of Example 6 using 2,3-dihydro-benzo[1,4]dioxine-6-carbaldehyde. [M+H]⁺386.86; ¹H-NMR (400 MHz, CDCl₃) δ 8.30 (s, 1H), 7.64 (s, 1H), 7.27 (s, 1H), 7.08 (s, 1H), 6.79 (d, 1H), 6.75 (d, 1H), 4.24 (s, 4H), 3.67 (s, 3H), 2.67 (t, 2H), 1.87 (t, 4H).

### EXAMPLE 13

### N′-(2,4-Dimethoxy-benzyl)-N-(3-imidazol-1-yl-[1,2,4]thiadiazol-5-yl)-N-methyl-propane-1,3-diamine

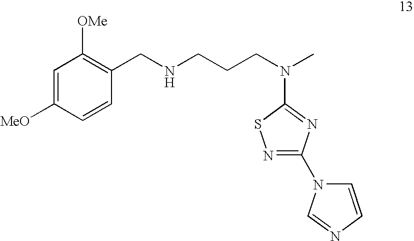 Example 13 was prepared following the procedures described in preparation of Example 6 using 2,4-dimethoxy-benzaldehyde. [M+H]⁺389.01; ¹H-NMR (400 MHz, CDCl₃) δ 8.30 (s, 1H), 7.64 (s, 1H), 7.08 (t, 2H), 6.43 (d, 1H), 6.40 (d, 1H), 3.81 (s, 6H), 3.74 (s, 2H), 3.12 (bs, 3H), 2.68 (t, 2H), 2.45 (bs, 2H), 1.92 (t, 2H).

### EXAMPLE 14

### N-(3-Imidazol-1-yl-[1,2,4]thiadiazol-5-yl)-N′-(1H-indol-5-ylmethyl)-N-methyl-propane-1,3-diamine

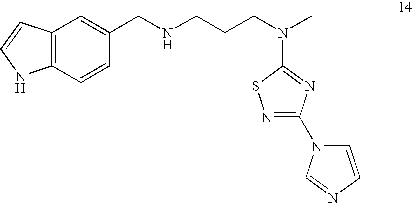

Example 14 was prepared following the procedures described in preparation of Example 6 using 1H-indole-5-carbaldehyde. [M+H]⁺368.04; ¹H-NMR (400 MHz, CDCl₃) δ 8.30 (s, 1H), 7.63 (s, 1H), 7.59 (s, 1H), 7.38 (d, 1H), 7.22 (d, 1H), 7.18 (d, 1H), 7.08 (s, 1H), 6.52 (d, 1H), 3.94 (s, 2H), 3.18 (bs, 3H), 2.78 (t, 2H), 1.98 (t, 2H).

### EXAMPLE 15

### N-(3-Imidazol-1-yl-[1,2,4]thiadiazol-5-yl)-N-methyl-N′-(1-methyl-1H-indol-2-ylmethyl)-propane-1,3-diamine

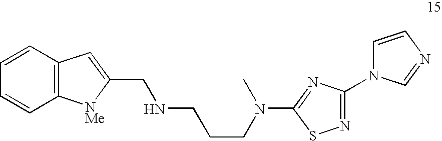 Example 15 was prepared following the procedures described in preparation of Example 6 using 1-methyl-1H-indole-2-carbaldehyde. [M+H]⁺382.07; ¹H-NMR (400 MHz, CDCl₃) δ 8.34 (s, 1H), 7.65 (s, 1H), 7.60 (d, 1H), 7.33 (d, 1H), 7.24 (t, 1H), 7.13 (t, 1H), 6.41 (s, 1H), 3.98 (s, 2H), 3.80 (s, 2H), 3.24 (bs, 3H), 2.78 (t, 2H), 1.92 (t, 2H).

### EXAMPLE 16

### N-(3-Imidazol-1-yl-[1,2,4]thiadiazol-5-yl)-N-methyl-N′-thiophen-2-ylmethyl-propane-1,3-diamine

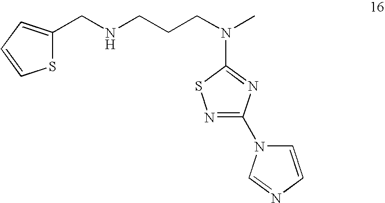 Example 16 was prepared following the procedures described in preparation of Example 6 using thiophene-2-carbaldehyde. [M+H]⁺335.11; ¹H-NMR (400 MHz, CDCl₃) δ 8.32 (s, 1H), 7.67 (s, 1H), 7.22 (dd, 1H), 7.11 (t, 1H), 6.97 (dd, 1H), 6.92 (s, 1H), 4.01 (s, 2H), 3.62 (bs, 2H), 3.08 (bs, 3H), 2.78 (t, 2H), 1.90 (t, 2H).

### EXAMPLE 17

### N-(3-Imidazol-1-yl-[1,2,4]thiadiazol-5-yl)-N-methyl-N′-pyridin-4-ylmethyl-propane-1,3-diamine

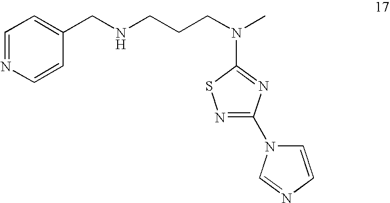 Example 17 was prepared following the procedures described in preparation of Example 6 using pyridine-4-carbaldehyde. [M+H]⁺330.05; ¹H-NMR (400 MHz, CDCl₃) δ 8.60-8.52 (m, 2H), 8.32 (s, 1H), 7.66 (d, 1H), 7.36-7.25 (m, 2H), 7.08 (d, 1H), 4.80 (s, 3H), 3.82 (m, 2H), 3.20 (s, 1H), 2.65 (t, 2H), 1.95 (m, 2H), 1.05 (m, 2H)

### EXAMPLE 18

### N′-(3,4-Dimethoxy-benzyl)-N-(3-imidazol-1-yl-[1,2,4]thiadiazol-5-yl)-N-methyl-propane-1,3-diamine

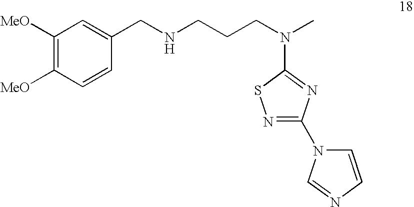 Example 18 was prepared following the procedures described in preparation of Example 6 using 3,4-dimethoxy-benzaldehyde. [M+H]⁺389.02; ¹H-NMR (400 MHz, CDCl₃) δ 8.33 (s, 1H), 7.66 (d, 1H), 7.30 (s, 1H), 7.11 (d, 1H), 6.91 (d, 1H), 6.83 (d, 1H), 3.90 (s, 3H), 3.88 (s, 3H), 3.76 (s, 2H), 3.15 (bs, 3H), 2.73 (t, 2H), 1.94 (t, 2H).

### EXAMPLE 19

### N′-Benzo[1,3]dioxol-5-ylmethyl-N-(3-imidazol-1-yl-[1,2,4]thiadiazo-5-yl)-N-methyl-propane-1,3-diamine

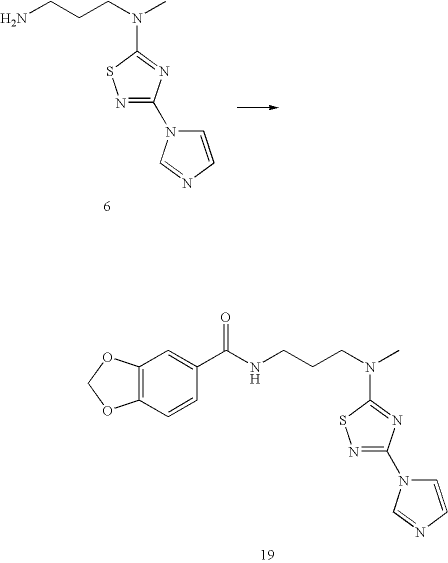 Piperonyloyl chloride (140 mg, 0.76 mmol) and DIEA (130 μL, 0.76 mmol) were added sequentially to a solution of N-1-(3-imidazol-1-yl-[1,2,4]thiadiazol-5-yl)-N-1-methyl-propane-1,3-diamine (150 mg, 0.63 mmol) in DCE (4 mL) at room temperature under nitrogen. The reaction mixture was stirred for 12 h prior to concentrating under vacuum. DCM (10 mL) was added and the solution was washed with NaHCO₃ (2×15 mL of a sat. aq. solution). The organic layer was concentrated under vacuum and purified by Prep-LC/MS to afford 102.1 mg (42%) of 19 as a white powder. [M+H]⁺387.03; ¹H NMR (400 MHz, CDCl3) δ 13.0 (s, 1H), 9.25 (s, 1H), 7.82 (d, 1H), 7.40 (d, 1H) 7.25 (d, 1H), 7.20 (s, 1H), 6.78 (d, 1H1), 6.01 (s, 2H), 3.48 (s, 2H), 3.15 (s, 2H), 3.12 (bs, 2H), 2.65 (s, 3H), 2.05 (m, 2H)

### EXAMPLE 20

### N-(2-Benzo[1,3]dioxol-5-yl-ethyl)-2-[(3-imidazol-1-yl-[1,2,4]thiadiazol-5-yl)-methyl-amino]-acetamide

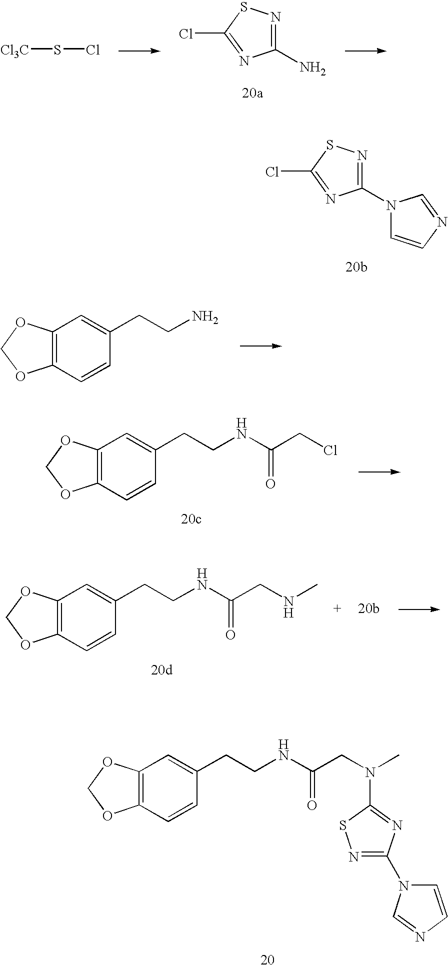 Step 1:
Preparation of Example 20a: 5-Chloro-[1,2,4]thiadiazol-3-ylamine

A solution of NaOH (43 g, 1.08 mmol) and water (43 mL) was added dropwise to a mixture trichloromethanesulfonyl chloride (20.0 g, 107 mmol), guanidine hydrochloride (10.2 g, 107 mmol) and DCM (200 mL) while maintaining an internal temperature of −10° C. to −20° C. The reaction was stirred at −10° C. for 3 h prior to warming to room temperature and stirring for 12 h. The resulting suspension was filtered through celite and the filtrate was transferred to a separatory funnel. The phases were separated and the aqueous layer was back extracted with DCM (100 mL). The combined organic layers were concentrated and the product was purified by flash chromatography (0-10% MeOH/DCM gradient) to give 2.15 g (4%) of 5-chloro-[1,2,4]thiadiazol-3-ylamine (20a). [M+H]⁺136.46.

Step 2:

Preparation of Example 20b: 5-Chloro-3-imidazol-1-yl-[1,2,4]thiadiazole

A mixture of 5-chloro-[1,2,4]thiadiazol-3-ylamine (1.00 g, 7.40 mmol), glyoxal 40% wt (5.35 g, 92.0 mmol) and ethanol (100 mL) were heated to 80° C. for 4 hours. Ammonium chloride (1.97 g, 37.0 mmol), formaldehyde (2.97 g, 37.0 mmol) and phosphoric acid (2.97 g, 30.0 mmol) were added and the reaction mixture was stirred at 80° C. for 14 hours. The mixture was cooled to room temperature and concentrated under vacuum. Water (50 mL) was added and the solution was washed with ethyl acetate (2×50 mL). The aqueous layer was brought to pH 8 with 1M NaOH and extracted with ethyl acetate (2×50 mL). The combined organic layers were concentrated under vacuum to give 200 mg (20%) of 5-chloro-3-imidazol-1-yl-[1,2,4]thiadiazole (20b). [M+H]⁺187.59.

Step 3:

Preparation of Example 20c: N-(2-Benzo[1,3]dioxol-5-yl-ethyl)-2-chloro-acetamide

Chloroacetyl chloride (48 μL, 0.60 mmol) was added to a solution of 3,4-methylenedioxyphenethylamine hydrochloride (100 mg, 0.50 mmol), TEA (70 μL) and DCE (2.0 mL) at room temperature under nitrogen. The reaction mixture was stirred for 12 h prior to concentrating under vacuum. DCM (10 mL) was added and the solution was washed with NaHCO₃ (2×15 mL of a sat. aq. solution). The organic layer was concentrated under vacuum and purified by Prep-LCMS to give 32 mg (27%) of N-(2-benzo[1,3]dioxol-5-yl-ethyl)-2-chloro-acetamide (20c). ¹H-NMR (400 MHz, CDCl₃) δ 6.75-6.62 (m, 3H), 5.92 (s, 2H), 4.01 (s, 2H), 3.52-3.47 (m, 2H), 2.79-2.72 (t, 2H).

Step 4:

Preparation of Example 20d: N-(2-Benzo[1,3]dioxol-5-yl-ethyl)-2-methylamino-acetamide

Methylamine (5.0 mL of a 33 wt. % solution in EtOH) was added to N-(2-benzo[1,3]dioxol-5-yl-ethyl)-2-chloro-acetamide (32 mg, 0.13 mmol) at room temperature under nitrogen. The solution was stirred for 16 h prior to concentrating under vacuum. The product was purified by prep-LCMS to give 21 mg (68%) of N-(2-benzo[1,3]dioxol-5-yl-ethyl)-2-methylamino-acetamide (20d). ¹H-NMR (400 MHz, CD₃OD) δ 6.73-6.65 (m, 3H), 5.89 (s, 2H), 3.46-3.39 (t, 2H), 3.31-3.29 (m, 2H), 2.74-2.70 (t, 2H), 2.35 (s, 3H).

Step 5:

Preparation of Example 20: N-(2-Benzo[1,3]dioxol-5-yl-ethyl)-2-[(3-imidazol-1-yl-[1,2,4]thiadiazol-5-yl)-methyl-amino]-acetamide was prepared following the procedures described for the preparation of Example 1, using 5-chloro-3-imidazol-1-yl-[1,2,4]thiadiazole instead of 3,5-dichlorothiadiazole. [M+H]⁺386.84; ¹H NMR (400 MHz, CDCl3) δ 8.24 (s, 1H), 7.62 (d, 1H), 7.12 (d, 1H), 6.62-6.48 (m, 3H), 6.00 (s, 1H), 5.82 (s, 2H), 4.18 (s, 2H), 3.45 (m, 2H), 3.12 (s, 3H), 2.65 (m, 2H).

### EXAMPLE 21

### N′-Benzo[1,3]dioxol-5-ylmethyl-N-(3-imidazol-1-yl-[1,2,4]thiadiazol-5-yl)-N-propyl-propane-1,3-diamine

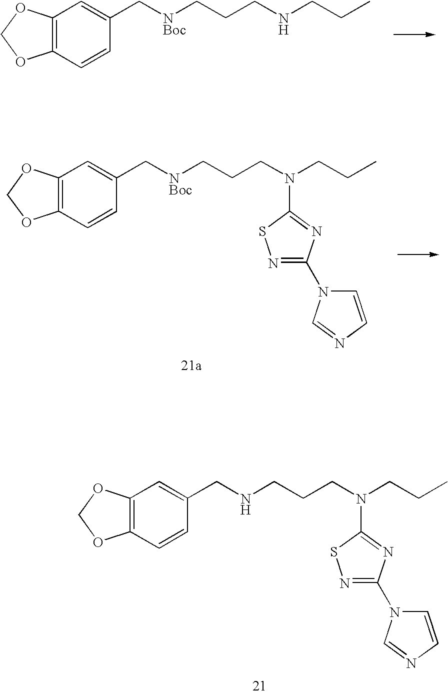 Step 1:
Preparation of Example 21a: Benzo[1,3]dioxol-5-ylmethyl-{3-[(3-imidazol-1-yl-[1,2,4]thiadiazol-5-yl)-propyl-amino]-propyl}-carbamic acid tert-butyl ester was prepared following the procedures described for the preparation of Example 1, using benzo[1,3]dioxol-5-ylmethyl-(3-propylamino-propyl)-carbamic acid tert-butyl ester and 5-chloro-3-imidazol-1-yl-[1,2,4]thiadiazole as the starting materials.
Step 2:
Preparation of example 21: N,N′-Benzo[1,3]dioxol-5-ylmethyl-N-(3-imidazol-1-yl-[1,2,4]thiadiazol-5-yl)-N-propyl-propane-1,3-diamine was prepared following the procedures described in preparation of Example 1. [M+H]⁺400.90; ¹H-NMR (400 MHz, CDCl₃) δ 8.28 (s, 1H), 7.62 (s, 1H), 7.04 (d, 1H), 6.80 (d, 1H), 5.92 (s, 2H), 3.64 (s, 2H), 2.64 (t, 2H), 1.88 (t, 3H), 1.71 (q, 2H), 1.60 (m, 2H), 0.99 (t, 4H).

### EXAMPLE 22

### N′-Benzo[1,3]dioxol-5-ylmethyl-N-cylopropyl-N-(3-imidazol-1-yl-[l,2,4]thiadiazol-5-yl)-propane-1,3-diamine

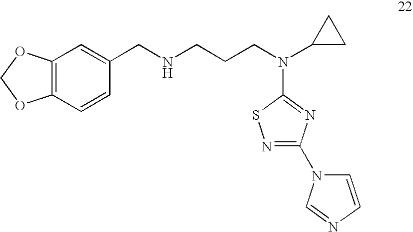 Example 22 was prepared following the procedures described in preparation of Example 1 using benzo[1,3]dioxol-5-ylmethyl-(3-cyclopropylamino-propyl)-carbamic acid tert-butyl ester. [M+H]⁺407.10; ¹H-NMR (400 MHz, CDCl₃) δ 8.26 (s, 1H), 7.62 (s, 1H), 7.07 (s, 1H), 7.79 (s, 1H), 6.70 (t, 2H), 5.93 (s, 2H), 3.74 (t, 2H), 3.63 (s, 2H), 2.72 (quin, 1H), 2.65 (2H, t), 1.93 (td, 2H), 1.29 (t, 2H), 0.92 (m, 2H).

### EXAMPLE 23

### 1-Benzo[1,3]dioxol-5-ylmethyl-4-(3-imidazol-1-yl-[1,2,4]thiadiazol-5-yl)-piperazine

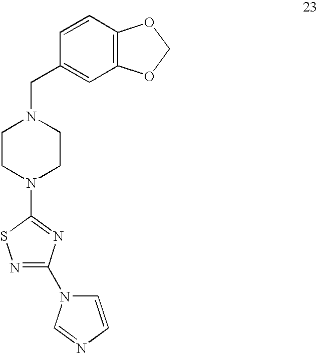 Example 23 was prepared following the procedures described in preparation of Example 1 using 1-benzo[1,3]dioxol-5-ylmethyl-piperazine. [M+H]⁺370.96; ¹H-NMR (400 MHz, CDCl₃) δ 8.30 (s, 1H), 7.65 (d, 1H), 7.09 (d, 1H), 6.87 (s, 1H), 6.75 (d, 2H), 5.97 (s, 2H), 3.57 (br s, 4H), 2.57 (t, 4H), 1.95 (s, 2H).

### EXAMPLE 24

### N-{3-[(3-Imidazol-1-yl-[1,2,4]thiadiazol-5-yl)-methyl-amino]-propyl}-4-trifluoromethoxy-benzenesulfonamide

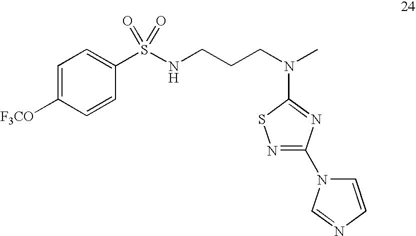 4-(Trifluoromethoxy)-benzenesulfonyl chloride (150 μL, 0.86 mmol) was added to a solution of N-1-(3-imidazol-1-yl-[1,2,4]thiadiazole-5-yl)-N-1-methyl-propane-1,3-diamine (100 mg, 0.43 mmol), DIEA (150 μL) and DCE (1 mL) at room temperature under nitrogen. The reaction mixture was stirred for 16 h prior to concentrating under vacuum. DCM (10 mL) was added and the solution was washed with NaHCO₃ (2×15 mL of a sat. aq. solution). The organic layer was concentrated under vacuum and purified by Prep-LCMS to give 34 mg (32%) of N-{3-[(3-imidazol-1-yl-[1,2,4]thiadiazol-5-yl)-methyl-amino]-propyl}-4-trifluoromethoxy-benzenesulfonamide (24). [M+H]⁺462.83; ¹H-NMR (400 MHz, CDCl₃) δ9.38 (br s, 1H), 8.01 (d, 1H), 7.91 (d, 2H), 7.43 (s, 1H), 7.36 (d, 2H), 3.30 (s, 3H), 3.08 (t, 2H), 2.00 (t, 2H).

### EXAMPLE 25

### Acetic acid 4-[((4-acetoxy-benzyl)-{3-[(3-imidazol-1-yl-[1,2,4]thiadiazol-5-yl)-methyl-amino]-propyl}-amino)-methyl]-phenyl Ester

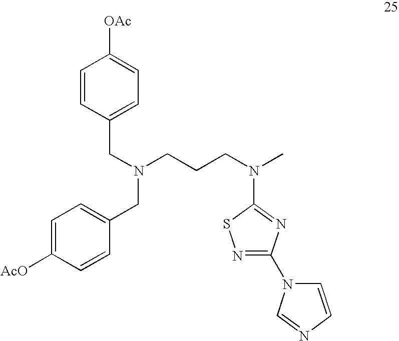

p-Toluenesulfonic acid (15 mg, 0.090 mmol) was added to a solution of 4-acetoxybenzaldehyde (80 mg, 0.49 mmol), 5 (120 mg, 0.50 mmol) and THF (2.0 mL) at room temperature under nitrogen. The mixture was heated to 80° C. for 30 minutes prior to cooling to room temperature. Glacial acetic acid (0.3 mL) and sodium triacetoxyborohydride (800 mg, 3.8 mmol) were added and the reaction mixture was stirred at room temperature for 14 h. The solution was concentrated under vacuum, water (10 mL) was added and the pH was adjusted to 9 with the addition of Na₂CO₃ (sat. aq. solution). The solution was extracted with ethyl acetate, washed with brine and the organic layer was concentrated under vacuum. The product was purified by Prep-LCMS to give 26 mg (10%) of acetic acid 4-[((4-acetoxy-benzyl)-{3-[(3-imidazol-1-yl-[1,2,4]thiadiazol-5-yl)-methyl-amino]-propyl}-amino)-methyl]-phenyl ester (25). [M+H]⁺536.32; ¹H-NMR (400 MHz, CDCl₃) δ 8.27 (s, 1H), 7.60 (s, 1H), 7.35 (d, 4H), 7.02 (m, 5H), 3.55 (s, 4H), 3.42 (br s, 2H), 2.95 (br s, 2H), 2.48 (t, 2H), 2.29 (s, 6H), 1.83 (t, 2H,).

### EXAMPLE 26

### N-(3-Imidazol-1-yl-[1,2,4]thiadiazol-5-yl)-N′,N′-bis-(4-hydroxy-benzyl)-N-methyl-propane-1,3-diamine

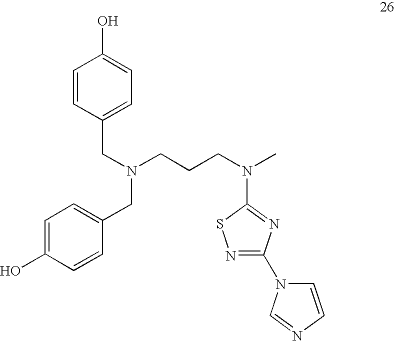 Example 26 was prepared following the procedures described in preparation of Example 25. [M+H]⁻450.99; ¹H-NMR (400 MHz, CD₃OD) δ 8.28 (s, 1H), 7.65 (s, 1H), 7.10 (d, 4H), 7.03 (s, 1H), 6.72 (d, 4H), 3.36 (m, 7H), 2.95 (br s, 2H), 2.38 (m, 2H), 1.77 (m, 2H).

### EXAMPLE 27

### (1-Benzo[1,3]dioxol-5-ylmethyl-pyrrolidin-3-ylmethyl)-(3-imidazol-1-yl-[1,2,4]thiadiazol-5-yl)-methyl-amine

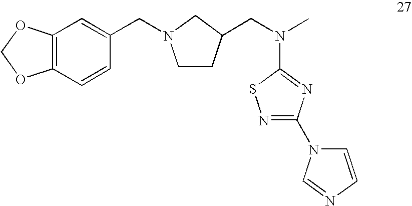 Example 27 was prepared following the procedures described in the preparation of Example 1 in Step 4 using (1-benzo[1,3]dioxol-5-ylmethyl-pyrrolidin-3-ylmethyl)-methyl-amine and 5-chloro-3-imidazol-1-yl-[1,2,4]thiadiazole. [M+H]⁺398.99; ¹H NMR (400 MHz, CDCl₃) δ 8.28 (s, 1H), 7.63 (s, 1H), 7.06 (s, 1H), 6.81 (s, 1H), 6.70-6.67 (m, 2H), 5.89 (s, 2H), 3.14 (s, 2H), 2.92 (s, 2H), 2.90 (m, 2H), 2.65-2.47 (m, 2H), 2.58 (s, 3H), 2.00 (m, 2H), 1.72 (m, 1H).

### EXAMPLE 28

### N′-Benzo[1,3]dioxol-5-ylmethyl-N-(3-imidazol-1-yl-[1,2,4]thiadiazo-5-yl)-N-methyl-ethane-1,2-diamine

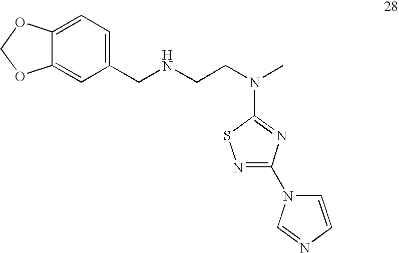 A solution of benzo[1,3]dioxol-5-ylmethyl-(2-methylamino-ethyl)-carbamic acid tert-butyl ester (200 mg, 649 μmol), 5-chloro-3-imidazol-1-yl-[1,2,4]thiadiazole (121 mg, 649 1mol), TEA (181 μL, 1.30 mmol) and DMSO (6 mL) was stirred at room temperature for 21 h. Water (50 mL) was added and the solution was extracted with ethyl acetate (2×25 mL). The combined organic layers were concentrated and the residue was dissolved in a mixture of TFA/DCM (4 mL of a 1:1 mixture). The solution was stirred at room temperature for 30 min prior to concentrating under vacuum. EtOAc (50 mL) was added and the solution was washed with 1M NaOH (50 mL) and brine (50 mL). The organic layer was concentrated and the product was purified using column chromatography (CH₂Cl₂ to 4:1 CH₂Cl₂/MeOH) to give 21.3 mg (9%) of N′-benzo[1,3]dioxol-5-ylmethyl-N-(3-imidazol-1-yl-[1,2,4]thiadiazol-5-yl)-N-methyl-ethane-1,2-diamine as a white solid. [M+H]⁺358.92; ¹H NMR (400 MHz, CDCl₃) δ 8.30 (s, 1H), 7.64 (s, 1H), 7.07 (s, 1H), 6.81 (s, 1H), 6.73 (m, 2H), 5.92 (s, 2H), 3.76 (s, 3H), 3.65 (br s, 1H), 3.14 (m, 4H), 2.95 (t, 2H).

### EXAMPLE 29

### N′-Benzo[1,3]dioxol-5-ylmethyl-N-(3-imidazol-1-yl-[1,2,4]thiadiazo-5-yl)-N-methyl-butane-1,4-diamine

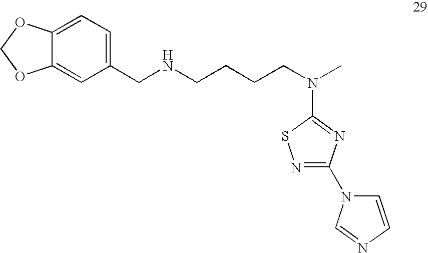 Example 29 was prepared following the procedures described in the preparation of Example 28 using benzo[1,3]dioxol-5-ylmethyl-(4-methylamino-butyl)-carbamic acid tert-butyl ester. [M+H]⁺386.90; ¹H NMR (400 MHz, d₆-DMSO) δ 8.34 (s, 1H), 7.75 (s, 1H), 7.11 (s, 1H), 6.93 (s, 1H), 6.86-6.78 (m, 2H), 6.00 (s, 2H), 3.63 (s, 3H), 3.36 (s, 2H), 3.20-3.05 (m, 5H), 1.72 (dtt, 2H), 1.49 (tt, 2H).

### EXAMPLE 30

### N′-Benzo[1,3]dioxol-5-ylmethyl-N-(3-imidazol-1-yl-[1,2,4]thiadiazo-5-yl)-N-thiazol-2-ylmethyl-propane-1,3-diamine

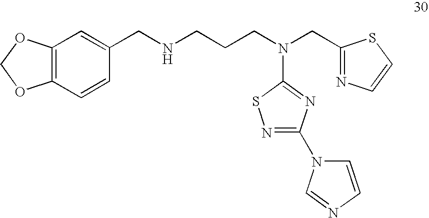 Example 30 was prepared following the procedures described in the preparation of Example 28 using benzo[1,3]dioxol-5-ylmethyl-{3-[(thiazol-2-ylmethyl)-amino]-propyl}-carbamic acid tert-butyl ester. [M+H]⁺455.87; ¹H NMR (400 MHz, CDCl₃) δ 8.33 (s, 1H), 7.74 (d, 1H), 7.66 (s, 1H), 7.33 (d, 1H), 7.09 (s, 1H), 6.83 (s, 1H), 6.73 (m, 2H), 5.92 (s, 2H), 5.03 (br s, 2H), 3.70 (s, 2H), 3.68 (br s, 1H), 3.61 (m, 2H), 2.68 (t, 2H), 1.95 (m, 2H).

### EXAMPLE 31

### N′-Benzo[1,3]dioxol-5-ylmethyl-N-(3-imidazol-1-yl-[1,2,4]thiadiazo-5-yl)-N-thiophen-2-ylmethyl-propane-1,3-diamine

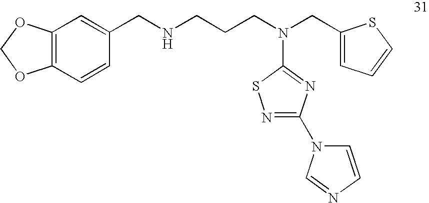 Example 31 was prepared following the procedures described in the preparation of Example 28 using benzo[1,3]dioxol-5-ylmethyl-{3-[(thiophen-2-ylmethyl)-amino]-propyl}-carbamic acid tert-butyl ester. [M+H]⁺455.27; ¹H NMR (400 MHz, CDCl₃) δ 8.34 (s, 1H), 7.67 (s, 1H), 7.28 (d, 1H), 7.09 (s, 1H), 7.07 (d, 1H), 6.97 (dd, 1H), 6.86 (s, 1H), 6.78-6.72 (m, 2H), 5.92 (s, 2H), 4.83 (s, 2H), 3.72 (s, 2H), 3.57 (m, 2H), 2.72 (t, 2H), 1.96 (m, 2H).

### EXAMPLE 32

### N′-Benzo[1,3]dioxol-5-ylmethyl-N-furan-2-ylmethyl-N-(3-imidazol-1-yl-[1,2,4]thiadiazol-5-yl)-propane-1,3-diamine

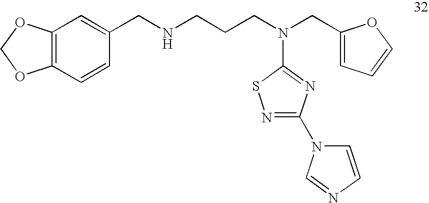

Example 32 was prepared following the procedures described in the preparation of Example 28 using benzo[1,3]dioxol-5-ylmethyl-{3-[(furan-2-ylmethyl)-amino]-propyl}-carbamic acid tert-butyl ester as the starting material. [M+H]⁺439.40; ¹H NMR (400 MHz, CDCl₃) δ 8.31 (s, 1H), 7.64 (s, 1H), 7.39 (d, 1H), 7.08 (s, 1H), 6.85 (s, 1H), 6.75-6.72 (m, 2H), 6.39-6.34 (m, 2H), 5.93 (s, 2H), 4.60 (s, 2H), 3.73 (s, 2H), 3.61 (s, 2H), 2.70 (t, 2H), 1.95 (m, 2H).

### EXAMPLE 33

### N′-(3-Imidazol-1-yl-[1,2,4]thiadiazol-5-yl)-N-thiazol-2-ylmethyl-propane-1,3-diamine

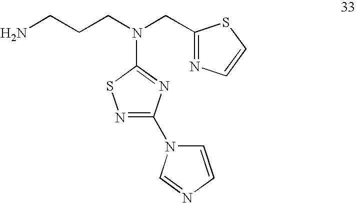 Example 33 was prepared following the procedures described in the preparation of Example 28 using {3-[(thiazol-2-ylmethyl)-amino]-propyl}-carbamic acid tert-butyl ester. [M+H]⁺321.94; ¹H NMR (400 MHz, CDCl₃) δ 7.85 (s, 1H), 7.81 (d, 1H), 7.46 (s, 1H), 7.41 (d, 1H), 7.31 (s, 1H), 4.78 (s, 2H), 4.27 (br s, 1H), 3.72 (t, 2H), 3.49 (t, 2H), 3.09 (br s, 1H), 2.20 (m, 2H).

### EXAMPLE 34

### [[3-(Benzo[1,3]dioxol-5-ylmethyl-tert-butoxycarbonyl-amino)-propyl]-(3-imidazo-1-yl-[1,2,4]thiadiazol-5-yl)-amino]-acetic Acid Ethyl Ester

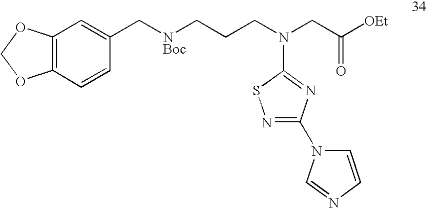 A solution of [3-(benzo[1,3]dioxol-5-ylmethyl-tert-butoxycarbonyl-amino)-propylamino]-acetic acid ethyl ester (1.20 g, 3.04 mmol), 5-chloro-3-imidazol-1-yl-[1,2,4]thiadiazole (568 mg, 3.04 mmol), TEA (1.27 mL, 9.13 mmol) and DMSO (15 mL) was stirred at room temperature for 17 h. Water (200 mL) was added and the solution was extracted with ethyl acetate (2×150 mL). The combined organic layers were washed with brine (200 mL) and concentrated to give 1.48 g (90%) of [[3-(benzo[1,3]dioxol-5-ylmethyl-tert-butoxycarbonyl-amino)-propyl]-(3-imidazol-1-yl-[1,2,4]thiadiazol-5-yl)-amino]-acetic acid ethyl ester. [M+H]⁺545.01.

### EXAMPLE 35

### [{3-[(Benzo[1,3]dioxol-5-ylmethyl)-amino]-propyl}-(3-imidazol-1-yl-[1,2,4]thiadiazol-5-yl)-amino]-acetic Acid Ethyl Ester

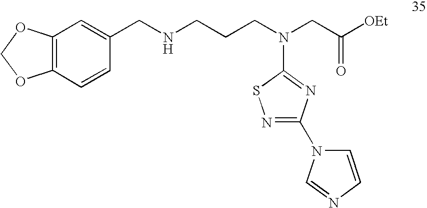 Example 35 was prepared following the procedures described in the preparation of Example 1 using [[3-(benzo[1,3]dioxol-5-ylmethyl-tert-butoxycarbonyl-amino)-propyl]-(3-imidazol-1-yl-[1,2,4]thiadiazol-5-yl)-amino]-acetic acid ethyl ester. [M+H]⁺444.95; ¹H NMR (400 MHz, CDCl₃) δ 8.27 (s, 1H), 7.63 (s, 1H), 7.09 (s, 1H), 6.81 (s, 1H), 6.74 (m, 2H), 5.94 (s, 2H), 4.27 (br s, 2H), 4.24 (q, 2H), 3.71 (s, 2H), 3.56 (br s, 2H), 2.74 (t, 2H), 1.90 (m, 2H), 1.30 (t, 3H).

### EXAMPLE 36

### 2-[{3-[(Benzo[1,3]dioxol-5-ylmethyl)-amino]-propyl}-(3-imidazol-1-yl-[1,2,4]thiadiazol-5-yl)-amino]-acetamide

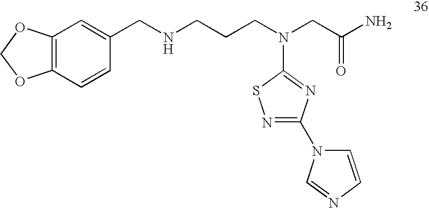

A solution of [[3-(benzo[1,3]dioxol-5-ylmethyl-tert-butoxycarbonyl-amino)-propyl]-(3-imidazo-1-yl-[1,2,4]thiadiazol-5-yl)-amino]-acetic acid ethyl ester (115 mg, 211 μmol) and NH₃ (10 mL of a 2.0 M solution in MeOH) was stirred at room temperature for 24 h. The reaction mixture was concentrated under vacuum and a 1:1 mixture of TFA/DCM (3 mL) was added. The solution was stirred at room temperature for 30 minutes prior to concentrating under vacuum. EtOAc (50 mL) was added and the solution was washed with 1M NaOH (50 mL) and brine (50 mL). The organic layer was concentrated and the product was purified using column chromatography (CH₂Cl₂ to 4:1 CH₂Cl₂/MeOH) to afford 53 mg (60%) of 2-[{3-[(benzo[1,3]dioxol-5-ylmethyl)-amino]-propyl}-(3-imidazol-1-yl-[1,2,4]thiadiazol-5-yl)-amino]-acetamide as a white solid. [M+H]⁺415.97; ¹H NMR (400 MHz, d₆-DMSO) δ 8.29 (s, 1H), 7.72 (s, 1H), 7.63 (br s, 1H), 7.28 (br s, 1H), 7.09 (s, 1H), 6.92 (s, 1H), 6.82 (m, 2H), 5.98 (s, 2H), 4.20 (br s, 1H), 3.60 (s, 2H), 3.33 (s, 2H), 2.54 (m, 4H), 1.80 (m, 2H).

### EXAMPLE 37

### [{3-[(Benzo[1,3]dioxol-5-ylmethyl)-amino]-propyl}-(3-imidazol-1-yl-[1,2,4]thiadiazol-5-yl)-amino]-acetic Acid

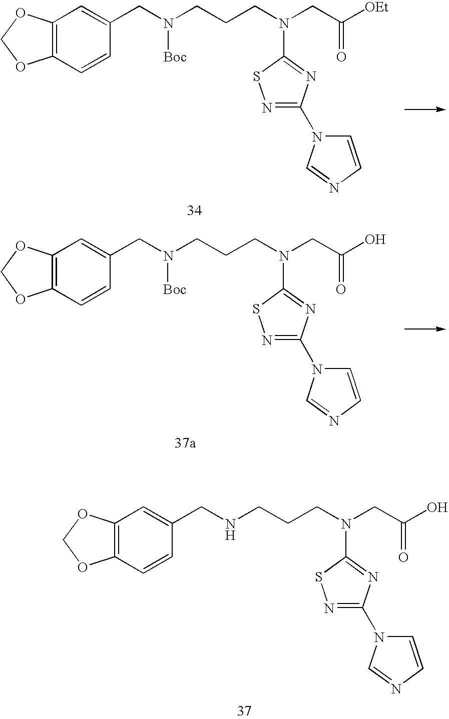 Step 1:
Preparation of Example 37a: [[3-(Benzo[1,3]dioxol-5-ylmethyl-tert-butoxycarbonyl-amino)-propyl]-(3-imidazol-1-yl-[1,2,4]thiadiazol-5-yl)-amino]-acetic Acid

A solution of [[3-(benzo[1,3]dioxol-5-ylmethyl-tert-butoxycarbonyl-amino)-propyl]-(3-imidazo-1-yl-[1,2,4]thiadiazol-5-yl)-amino]-acetic acid ethyl ester (489 mg, 898 μmol), 1.0M aqueous LiOH (1.35 mL, 1.35 mmol) and THF (10 mL) was stirred at room temperature for 18 h. The reaction mixture was concentrated under vacuum to afford a white solid. EtOAc (100 mL) was added and the solution was washed with 1.0M aqueous HCl (50 mL) and brine (50 mL). The organic layer was concentrated to give 452 mg (97%) of [[3-(benzo[1,3]dioxol-5-ylmethyl-tert-butoxycarbonyl-amino)-propyl]-(3-imidazo-1-yl-[1,2,4]thiadiazol-5-yl)-amino]-acetic acid (37a) as a white solid. [M+H]⁻517.01.

Step 2:

Preparation of Example 37: [{3-[(Benzo[1,3]dioxol-5-ylmethyl)-amino]-propyl}-(3-imidazol-1-yl-[1,2,4]thiadiazol-5-yl)-amino]-acetic acid was prepared following the procedures described in preparation of Example 1. [M+H]⁺416.92; ¹H NMR (400 MHz, d₆-DMSO) δ 9.05 (s, 1H), 8.79 (br s, 1H), 8.00 (s, 1H), 7.49 (s, 1H), 7.06 (s, 1H), 6.97 (m, 2H), 6.06 (s, 2H), 4.39 (br s, 2H), 4.26 (s, 2H), 3.66 (m, 2H), 3.00 (m, 2H), 2.01 (m, 2H).

### EXAMPLE 38

### 2-[{3-[(Benzo[1,3]dioxol-5-ylmethyl)-amino]-propyl}-(3-imidazol-1-yl-[1,2,4]thiadiazol-5-yl)-amino]-N-methyl-acetamide

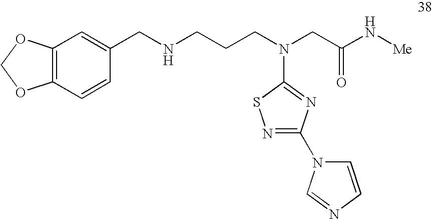 Example 38 was prepared following the procedures described for the preparation of Example 36 using MeNH₂. [M+H]⁺429.99; ¹H NMR (400 MHz, d₆-DMSO) δ 8.31 (s, 1H), 8.00 (br s, 1H), 7.72 (s, 1H), 7.10 (s, 1H), 6.93 (s, 1H), 6.82 (m, 2H), 5.99 (s, 2H), 3.98 (s, 2H), 3.64 (br s, 2H), 3.19 (t, 2H), 2.99 (s, 3H), 2.88 (t, 2H), 2.54 (br s, 1H), 1.84 (m, 2H).

### EXAMPLE 39

### 2-[{3-[(Benzo[1,3]dioxol-5-ylmethyl)-amino]-propyl}-(3-imidazol-1-yl-[1,2,4]thiadiazol-5-yl)-amino]-N,N-dimethyl-acetamide

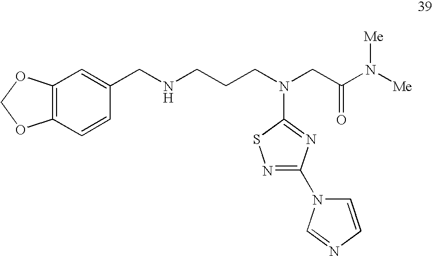 Example 39 was prepared following the procedures described for the preparation of Example 36 using [[3-(benzo[1,3]dioxol-5-ylmethyl-tert-butoxycarbonyl-amino)-propyl]-(3-imidazo-1-yl-[1,2,4]thiadiazol-5-yl)-amino]-acetic acid and Me₂NH as the starting materials. [M+H]⁺443.99; ¹H NMR (400 MHz, d₆-DMSO) δ 8.34 (s, 1H), 7.75 (s, 1H), 7.12 (s, 1H), 6.99 (s, 1H), 6.87 (m, 2H), 6.03 (s, 2H), 4.51 (br s, 1H), 3.78 (br s, 2H), 3.38 (br s, 2H), 3.05 (s, 3H), 2.89 (s, 3H), 2.75 (m, 2H), 2.53 (m, 2H), 1.89 (m, 2H).

### EXAMPLE 40

### [[3-(Benzo[1,3]dioxol-5-ylmethyl-methyl-amino)-propyl]-(3-imidazol-1-yl-[1,2,4]thiadiazol-5-yl)-amino]-acetic Acid Ethyl Ester

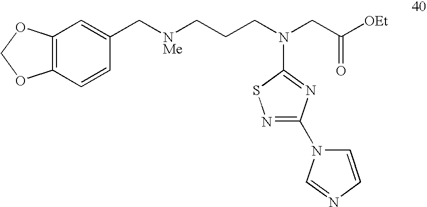 Example 40 was prepared following the procedures described in the preparation of Example 1 using [3-(benzo[1,3]dioxol-5-ylmethyl-methyl-amino)-propylamino]-acetic acid ethyl ester. [M+H]⁺458.98; ¹H NMR (400 MHz, CDCl₃) δ 8.26 (s, 1H), 7.63 (s, 1H), 7.07 (s, 1H), 6.82 (s, 1H), 6.73 (m, 2H), 5.93 (s, 2H), 4.28 (s, 2H), 4.23 (q, 2H), 3.52 (s, 2H), 3.43 (m, 2H), 2.46 (t, 2H), 2.21 (s, 3H), 1.91 (m, 2H), 1.29 (t, 3H).

### EXAMPLE 41

### 2-[[3-(Benzo[1,3]dioxol-5-ylmethyl-methyl-amino)-propyl]-(3-imidazo-1-yl-[1,2,4]thiadiazol-5-yl)-amino]-acetamide

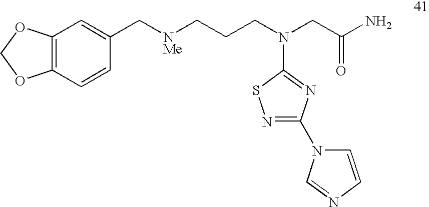 Example 41: was prepared following the procedures described in the preparation of Example 36 using [[3-(benzo[1,3]dioxol-5-ylmethyl-methyl-amino)-propyl]-(3-imidazol-1-yl-[1,2,4]thiadiazol-5-yl)-amino]-acetic acid ethyl ester and NH₃. [M+H]⁺429.87; ¹H NMR (400 MHz, d₆-DMSO) δ 8.24 (s, 1H), 7.68 (s, 1H), 7.54 (br s, 1H), 7.18 (br s, 1H), 7.06 (s, 1H), 6.86-6.70 (m, 3H), 5.98 (s, 2H), 3.45-3.35 (m, 4H), 3.31 (s, 2H), 2.36 (m, 2H), 2.10 (s, 31H), 1.72 (m, 2H).

### EXAMPLE 42

### {3-[(Benzo[1,3]dioxol-5-ylmethyl)-amino]-propyl}-(3-imidazol-1-yl-[1,2,4]thiadiazol-5-yl)-amino]-acetonitrile

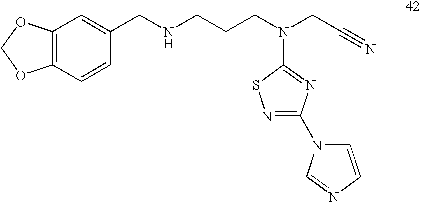 Example 42 was prepared following the procedures described in the preparation of Example 28 using benzo[1,3]dioxol-5-ylmethyl-[3-(cyanomethyl-amino)-propyl]-carbamic acid tert-butyl ester. [M+H]⁺397.90; ¹H NMR (400 MHz, CDCl₃) δ 8.32 (s, 1H), 7.67 (s, 1H), 7.11 (s, 1H), 6.81 (s, 1H), 6.80-6.73 (m, 2H), 5.94 (s, 2H), 4.58 (s, 2H), 3.69 (s, 2H), 3.60 (t, 2H), 2.72 (t, 2H), 1.92 (tt, 2H).

### EXAMPLE 43

### N′-Benzo[1,3]dioxol-5-ylmethyl-N-(3-imidazol-1-yl-[1,2,4]thiadiazol-5-yl)-N-(1H-tetrazol-5-ylmethyl)-propane-1,3-diamine

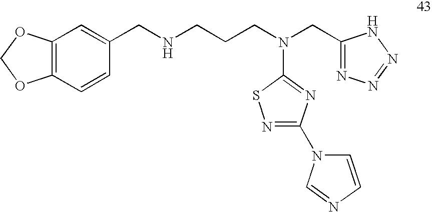 A suspension of [1{3-[(benzo[1,3]dioxol-5-ylmethyl)-amino]-propyl}-(3-imidazol-1-yl-[1,2,4]thiadiazol-5-yl)-amino]-acetonitrile 42 (37 mg, 91 μmol), sodium azide (12 mg, 184 μmol), zinc (II) bromide (10 mg, 46 μmol) in a 2:1 mixture of H₂O/PrOH (1 mL) was heated to 150° C. for 24 h. The reaction was cooled to room temperature prior to loading directly onto a SiO₂ column (CH₂Cl₂ to 4:1 CH₂Cl₂/MeOH) to give 14 mg (35%) of N-benzo[1,3]dioxol-5-ylmethyl-N-(3-imidazol-1-yl-[1,2,4]thiadiazol-5-yl)-N-(1H-tetrazol-5-ylmethyl)-propane-1,3-diamine as a white solid. [M+H]441.01; ¹H NMR (400 MHz, CD₃OD) δ 8.35 (s, 1H), 7.74 (s, 1H), 7.07 (s, 1H), 6.85-6.74 (m, 3H), 5.92 (s, 2H), 4.43 (s, 2H), 4.06 (s, 2H), 3.81 (br s, 2H), 3.31 (t, 2H), 3.06 (t, 2H), 2.17 (tt, 2H).

### EXAMPLE 44

### 2-[{3-[(Benzo[1,3]dioxol-5-ylmethyl)-amino]-propyl}-(3-imidazol-1-yl-[1,2,4]thiadiazol-5-yl)-amino]-acetamidine N′-methyl-hydrazide

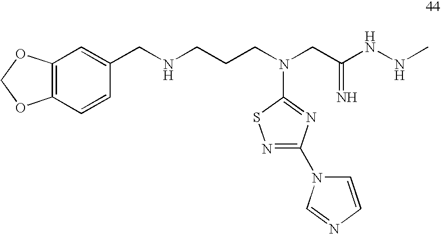 Methylhydrazine (37 μL, 700 μmol) was added to a solution of [{3-[(benzo[1,3]dioxol-5-ylmethyl)-amino]-propyl}-(3-imidazol-1-yl-[1,2,4]thiadiazol-5-yl)-amino]-acetonitrile (27 mg, 70 μmol) in EtOH (500 μL) at room temperature under nitrogen. The reaction mixture stirred at for 19 h prior to concentrating under vacuum. The product was purified using column chromatography (CH₂Cl₂ to 4:1 CH₂Cl₂/MeOH) to give 14 mg (45%) of 2-[{3-[(benzo[1,3]dioxol-5-ylmethyl)-amino]-propyl}-(3-imidazol-1-yl-[1,2,4]thiadiazol-5-yl)-amino]-acetamidine N-methyl-hydrazide as a white solid. [M+H]⁺444.53; ¹H NMR (400 MHz, CD₃OD) δ 8.34 (s, 1H), 7.73 (s, 1H), 7.07 (s, 1H), 6.83 (s, 1H), 6.77-6.72 (m, 2H), 5.88 (s, 2H), 4.23 (br s, 2H), 3.70 (d, 3H), 3.68 (s, 2H), 3.61 (s, 2H), 3.30 (s, 2H), 2.72 (m, 2H), 1.96 (tt, 2H).

### EXAMPLE 45

### 2-[{3-[(Benzo[1,3]dioxol-5-ylmethyl)-amino]-propyl-(3-imidazol-1-yl-[1,2,4]thiadiazol-5-yl)-amino]-N-hydroxy-acetamidine

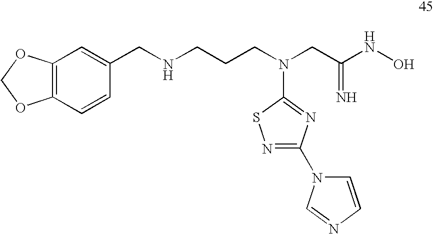 Hydroxylamine (1.0 mL of a 50 wt % in H₂O, 15 mmol) was to a solution of [{3-[(benzo[1,3]dioxol-5-ylmethyl)-amino]-propyl}-(3-imidazol-1-yl-[1,2,4]thiadiazol-5-yl)-amino]-acetonitrile (30 mg, 75 μmol) in MeOH (1.0 mL) at room temperature. The reaction mixture stirred for 30 h prior to concentrating to a white solid. The product was purified using column chromatography (CH₂Cl₂ to 4:1 CH₂Cl₂/MeOH) to give 7.0 mg (22%) of 2-[{3-[(benzo[1,3]dioxol-5-ylmethyl)-amino]-propyl}-(3-imidazol-1-yl-[1,2,4]thiadiazol-5-yl)-amino]-N-hydroxy-acetamidine as a white solid. [M+H]⁺430.91; ¹H NMR (400 MHz, CD₃OD) δ 8.39 (s, 1H), 7.79 (s, 1H), 7.76 (s, 1H), 7.07 (s, 1H), 6.82 (s, 1H), 6.76-6.70 (m, 2H), 5.89 (s, 2H), 4.19 (s, 2H), 3.65 (s, 2H), 3.58 (m, 2H), 2.63 (t, 2H), 1.94 (tt, 2H).

### EXAMPLE 46

### N-Benzo[1,3]dioxol-5-ylmethyl-N′-(3-imidazol-1-yl-[1,2,4]thiadiazol-5-yl)-N,N′-dimethyl-propane-1,3-diamine

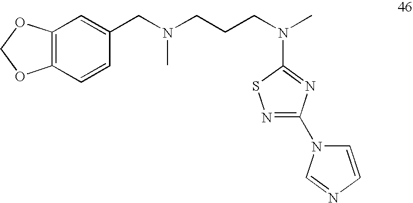 Example 46 was prepared following the procedures described in the preparation of Example 6 using N′-benzo[1,3]dioxol-5-ylmethyl-N-(3-imidazol-1-yl-[1,2,4]thiadiazol-5-yl)-N-methyl-propane-1,3-diamine and formalin. [M+H]⁺387.66; ¹H NMR (400 MHz, CDCl₃) δ 8.29 (s, 1H), 7.64 (t, 1H), 7.07 (t, 1H), 6.81 (s, 1H), 6.72 (m, 2H), 5.93 (s, 2H), 3.60-3.50 (s, 3H), 3.39 (s, 3H), 3.11 (br s, 2H), 2.40 (t, 2H), 2.18 (t, 2H), 1.87 (m, 2H).

### EXAMPLE 47

### Benzo[1,3]dioxol-5-ylmethyl-(3-[3-(4-iodo-imidazol-1-yl)-[1,2,4]thiadiazol-5-yl]-methyl-amino}-propyl)-carbamic Acid tert-butyl Ester

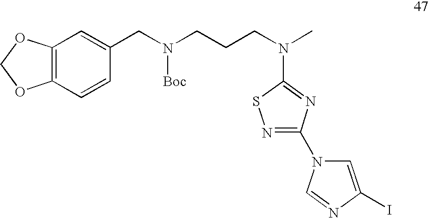 Example 47 was prepared following the procedures described in the preparation of Example 1 using 4-iodo-1H-imidazole. [M+H]⁺598.90.

### EXAMPLE 48

### N′-Benzo[1,3]dioxol-5-ylmethyl-N-[3-(4-iodo-imidazol-1-yl)-[1,2,4]thiadiazol-5-yl]-N-methyl-propane-1,3-diamine

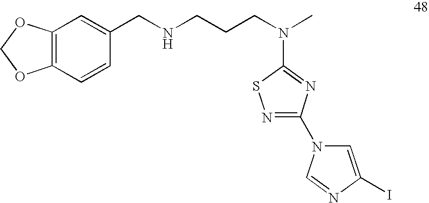 Example 48 was prepared following the procedures described in the preparation of Example 1 using benzo[1,3]dioxol-5-ylmethyl-(3-{[3-(4-iodo-imidazol-1-yl)-[1,2,4]thiadiazol-5-yl]-methyl-amino}-propyl)-carbamic acid tert-butyl ester. [M+H]⁺499.32; ¹H NMR (400 MHz, CDCl₃) δ 8.12 (s, 1H), 7.71 (s, 1H), 6.76 (s, 1H), 6.70 (m, 2H), 5.91 (s, 2H), 3.65 (s, 3H), 3.70-3.50 (br s, 2H), 3.09 (br s, 2H), 2.64 (t, 2H), 1.87 (m, 2H), 1.62 (br s, 1H).

### EXAMPLE 49

### N-(2,3-Dihydro-benzo [1,4]dioxin-6-ylmethyl)-N′-(3-imidazol-1-yl-[1,2,4]thiadiazol-5-yl)-N,N′-dimethyl-propane-1,3-diamine

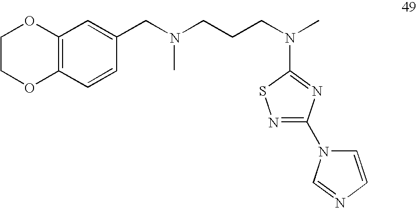 Example 49 was prepared following the procedures described in the preparation of Example 6 using N′-(2,3-dihydro-benzo[1,4]dioxin-6-ylmethyl)-N-(3-imidazol-1-yl-[1,2,4]thiadiazol-5-yl)-N-methyl-propane-1,3-diamine and formalin. [M+H]401.55; 1H NMR (400 MHz, CDCl₃) δ 8.30 (s, 1H), 7.64 (s, 1H), 7.10 (s, 1H), 6.80-6.75 (m, 3H), 4.24 (s, 4H), 3.67 (s, 3H), 3.14 (s, 3H), 2.67 (t, 2H), 1.87 (t, 4H).

### EXAMPLE 50

### N′-[2-(2,3-Dihydro-benzo[1,4]dioxin-6-yl)-ethyl]-N-(3-imidazol-1-yl-[1,2,4]thiadiazol-5-yl)-N-methyl-ethane-1,2-diamine

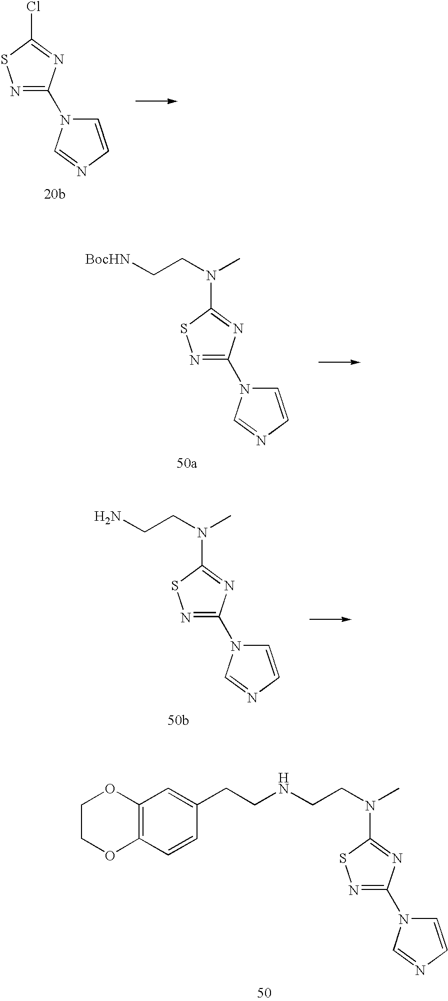 Step 1:
Preparation of example 50a: {2-[(3-Imidazol-1-yl-[1,2,4]thiadiazol-5-yl)-methyl-amino]-ethyl}-carbamic acid tert-butyl ester was prepared following the procedures described in the preparation of Example 1 using (2-methylamino-ethyl)-carbamic acid tert-butyl ester. [M+H]⁺325.12.
Step 2:
Preparation of example 50b: N-1-(3-Imidazol-1-yl-[1,2,4]thiadiazol-5-yl)-N-1-methyl-ethane-1,2-diamine was prepared following was prepared following the procedures described in the preparation of Example 1 using {2-[(3-imidazol-1-yl-[1,2,4]thiadiazol-5-yl)-methyl-amino]-ethyl}-carbamic acid tert-butyl ester. [M+H]⁺225.07.
Step 3:
Preparation of example 50: N′-[2-(2,3-Dihydro-benzo [1,4]dioxin-6-yl)-ethyl]-N-(3-imidazol-1-yl-[1,2,4]thiadiazol-5-yl)-N-methyl-ethane-1,2-diamine was prepared following the procedures described in the preparation of Example 6 using (2,3-dihydro-benzo[1,4]dioxin-6-yl)-acetaldehyde. [M+H]⁺387.74; ¹H NMR (400 MHz, CDCl₃) δ 8.36 (s, 1H), 7.64 (s, 1H), 7.06 (s, 1H), 6.73 (d, 1H), 6.66 (d, 1H), 6.61 (dd, 2H), 4.21 (s, 4H), 3.74 (br s, 2H), 3.13 (s, 3H), 3.06 (t, 2H), 2.98 (t, 2H), 2.78 (t, 2H).

### EXAMPLE 51

### N-[2-(2,3-Dihydro-benzo [1,4]dioxin-6-yl)-ethyl]-N′-(3-imidazol-1-yl-[1,2,4]thiadiazol-5-yl)-N,N′-dimethyl-ethane-1,2-diamine

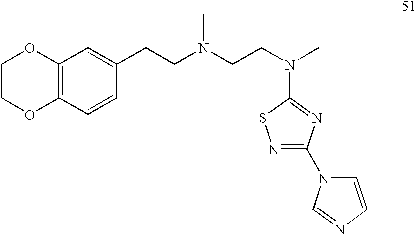 Example 51 was prepared following the procedures described in the preparation of Example 6 using N′-[2-(2,3-dihydro-benzo[1,4]dioxin-6-yl)-ethyl]-N-(3-imidazol-1-yl-[1,2,4]thiadiazol-5-yl)-N-methyl-ethane-1,2-diamine and formalin. [M+H]401.33; ¹H NMR (400 MHz, CD₃OD) 6 8.31 (s, 1H), 7.69 (s, 1H), 7.04 (s, 1H), 6.70-6.50 (m, 3H), 4.13 (s, 4H), 3.74 (br s, 2H), 3.03 (s, 3H), 2.68 (t, 2H), 2.60-2.50 (m, 4H), 2.32 (s, 3H).

### EXAMPLE 52

### N′-[2-(2,3-Dihydro-benzo [1,4]dioxin-6-yl)-ethyl]-N-(3-imidazol-1-yl-[1,2,4]thiadiazol-5-yl)-N-methyl-propane-1,3-diamine

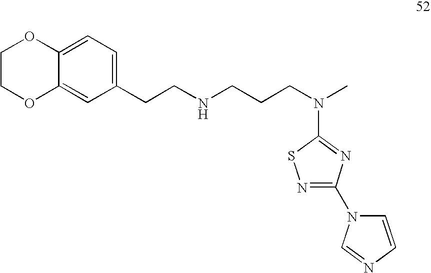 Example 52 was prepared following the procedures described in the preparation of Example 6 using 5 and (2,3-dihydro-benzo[1,4]dioxin-6-yl)-acetaldehyde as starting materials. [M+H]⁺401.29; ¹H NMR (400 MHz, CDCl₃) δ 8.33 (s, 1H), 7.65 (s, 1H), 7.08 (s, 1H), 6.76 (d, 1H), 6.68 (d, 1H), 6.63 (dd, 1H), 4.22 (s, 4H), 3.70-3.50 (br s, 2H), 3.11 (s, 3H), 2.89 (t, 2H), 2.80-2.70 (m, 4H), 1.97 (m, 2H).

### EXAMPLE 53

### N-[2-(2,3-Dihydro-benzo[1,4]dioxin-6-yl)-ethyl]-N′-(3-imidazol-1-yl-[1,2,4]thiadiazol-5-yl)-N,N′-dimethyl-propane-1,3-diamine

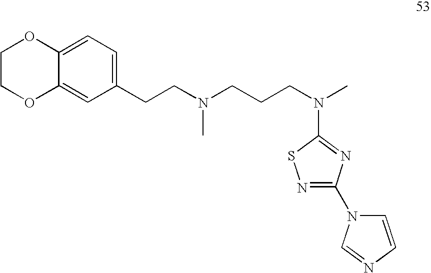 Example 53 was prepared following the procedures described in the preparation of Example 6 using N′-[2-(2,3-dihydro-benzo[1,4]dioxin-6-yl)-ethyl]-N-(3-imidazol-1-yl-[1,2,4]thiadiazol-5-yl)-N-methyl-propane-1,3-diamine and formalin as the starting materials. [M+H]⁺415.86; ¹H NMR (400 MHz, CDCl₃) δ 8.29 (s, 1H), 7.64 (s, 1H), 7.06 (s, 1H), 6.74 (d, 1H), 6.67 (d, 1H), 6.62 (dd, 1H), 4.19 (s, 4H), 3.60-3.30 (br s, 2H), 3.08 (s, 3H), 2.68-2.52 (m, 4H), 2.41 (t, 2H), 2.27 (s, 3H), 1.83 (m, 2H).

### EXAMPLE 54

### N′-(2-Benzo[1,3]dioxol-5-yl-ethyl)-N-(3-imidazol-1-yl-[1,2,4]thiadiazol-5-yl)-N-methyl-propane-1,3-diamine

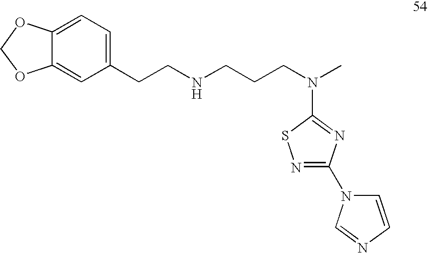 Example 54 was prepared following the procedures described in the preparation of Example 6 using 5 and benzo[1,3]dioxol-5-yl-acetaldehyde as starting materials. [M+H]⁺387.30; ¹H NMR (400 MHz, CDCl₃) δ 8.31 (s, 1H), 7.65 (s, 1H), 7.08 (s, 1H), 6.70 (d, 1H), 6.67 (d, 1H), 6.62 (dd, 1H), 5.91 (s, 2H), 3.70-3.50 (br s, 2H), 3.11 (s, 3H), 2.85 (t, 2H), 2.76-2.68 (m, 4H), 1.83 (m, 2H).

### EXAMPLE 55

### N-(2-Benzo[1,3]dioxol-5-yl-ethyl)-N′-(3-imidazol-1-yl-[1,2,4]thiadiazol-5-yl)-N,N′-dimethyl-propane-1,3-diamine

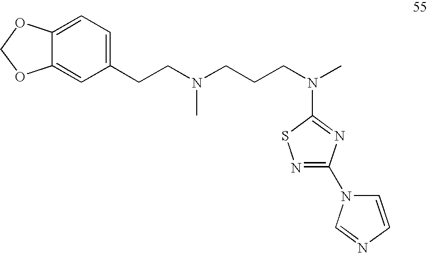 Example 55 was prepared following the procedures described in the preparation of Example 6 using N′-(2-benzo[1,3]dioxol-5-yl-ethyl)-N-(3-imidazol-1-yl-[1,2,4]thiadiazol-5-yl)-N-methyl-propane-1,3-diamine and formalin. [M+H]⁺401.30; ¹H NMR (400 MHz, CDCl₃) δ 8.28 (s, 1H), 7.63 (s, 1H), 7.06 (s, 1H), 6.69 (d, 1H), 6.65 (d, 1H), 6.60 (dd, 1H), 5.88 (s, 2H), 3.55-3.30 (br s, 2H), 3.08 (s, 3H), 2.66 (t, 2H), 2.55 (t, 2H), 2.42 (t, 2H), 2.27 (s, 3H), 1.81 (m, 2H).

### EXAMPLE 56

### N′-Benzo[1,3]dioxol-5-ylmethyl-N-benzofuran-5-ylmethyl-N-(3-imidazol-1-yl-[1,2,4]thiadiazol-5-yl)-propane-1,3-diamine

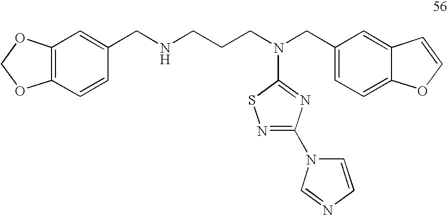 Example 56 was prepared following the procedures described in the preparation of Example 28 using benzo[1,3]dioxol-5-ylmethyl-{3-[(benzofuran-5-ylmethyl)-amino]-propyl}-carbamic acid tert-butyl ester. [M+H]⁺489.39.

### EXAMPLE 57

### N′-Benzo[1,3]dioxol-5-ylmethyl-N-(3-imidazol-1-yl-[1,2,4]thiadiazo-5-yl)-N-pyridin-3-ylmethyl-propane-1,3-diamine

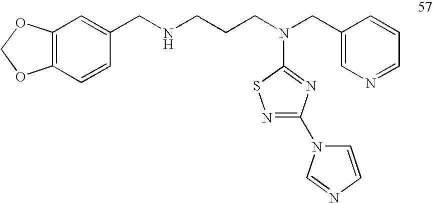 Example 57 was prepared following the procedures described in the preparation of Example 28 using benzo[1,3]dioxol-5-ylmethyl-{3-[(pyridin-3-ylmethyl)-amino]-propyl}-carbamic acid tert-butyl ester. [M+H]⁺450.55.

### EXAMPLE 58

### 4-{[{3-[(Benzo[1,3]dioxol-5-ylmethyl)-amino]-propyl}-(3-imidazol-1-yl-[1,2,4]thiadiazol-5-yl)-amino]-methyl}-phenol

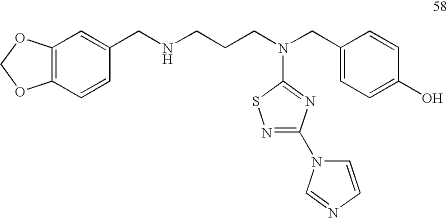 Example 58 was prepared following the procedures described in the preparation of Example 28 using benzo[1,3]dioxol-5-ylmethyl-[3-(4-hydroxy-benzylamino)-propyl]-carbamic acid tert-butyl ester. [M+H]⁺464.96.

### EXAMPLE 59

### N′-Benzo[1,3]dioxol-5-ylmethyl-N-(3-imidazol-1-yl-[1,2,4]thiadiazo-5-yl)-N-(4-methylsulfanyl-benzyl)-propane-1,3-diamine

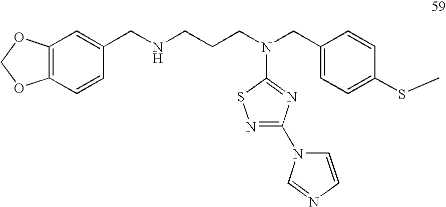 Example 59 was prepared following the procedures described in the preparation of Example 28 using benzo[1,3]dioxol-5-ylmethyl-[3-(4-methylsulfanyl-benzylamino)-propyl]-carbamic acid tert-butyl ester. [M+H]⁺495.59.

### EXAMPLE 60

### N-Benzo[1,3]dioxol-5-ylmethyl-N′-(4-dimethylamino-benzyl)-N′-(3-imidazol-1-yl-[1,2,4]thiadiazol-5-yl)-propane-1,3-diamine

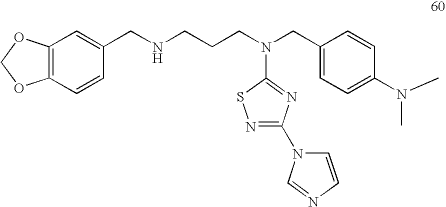 Example 60 was prepared following the procedures described in the preparation of Example 28 using benzo[1,3]dioxol-5-ylmethyl-[3-(4-dimethylamino-benzylamino)-propyl]-carbamic acid tert-butyl ester. [M+H]⁺492.64.

### EXAMPLE 61

### N′-Benzo[1,3]dioxol-5-ylmethyl-N-(3-imidazol-1-yl-[1,2,4]thiadiazol-5-yl)-N-(-methyl-1H-imidazol-4-ylmethyl)-propane-1,3-diamine

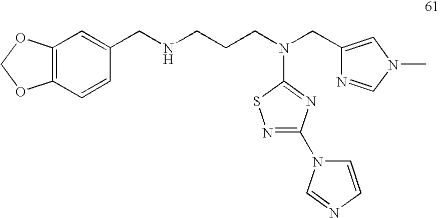 Example 61 was prepared following the procedures described in the preparation of Example 28 using benzo[1,3]dioxol-5-ylmethyl-{3-[(1-methyl-1 H-imidazol-4-ylmethyl)-amino]-propyl}-carbamic acid tert-butyl ester. [M+H]⁺453.58.

### EXAMPLE 62

### N-(2-Benzo[1,3]dioxol-5-yl-ethyl)-N-(3-imidazol-1-yl-[1,2,4]thiadiazol-5-yl)-N-methyl-ethane-1,2-diamine

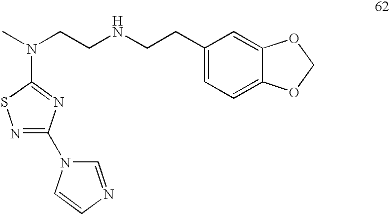 Example 62 was prepared following the procedures described in the preparation of Example 6 using N-1-(3-Imidazol-1-yl-[1,2,4]thiadiazol-5-yl)-N-1-methyl-ethane-1,2-diamine and benzo[1,3]dioxol-5-yl-acetaldehyde as the starting materials. [M+H]⁺373.23 ; ¹H NMR (400 MHz, CDCl₃) δ 8.30 (s, 1H), 7.62 (s, 1H), 7.04 (s, 1H), 6.65-6.56 (m, 3H), 5.90 (s, 2H), 3.60 (br s, 2H), 3.12 (m, 3H), 2.93 (m, 2H), 2.87 (m, 2H), 2.69 (m, 2H).

### EXAMPLE 63

### N-(2-Benzo[1,3]dioxol-5-yl-ethyl)-N′-(3-imidazol-1-yl-[1,2,4]thiadiazol-5-yl)-N,N′-dimethyl-ethane-1,2-diamine

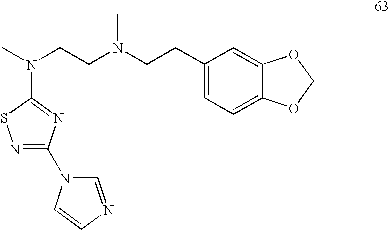 A mixture of AP-(2-Benzo[1,3]dioxol-5-yl-ethyl)-N-(3-imidazol-1-yl-[1,2,4]thiadiazol-5-yl)-N-methyl-ethane-1,2-diamine (438 mg, 1.20 mmol), formalin (500 μL of a 377% solution in water), methanol (8 mL) and acetic acid (1.0 mL) were stirred at room temperature for 15 minutes. Sodium triacetoxyborohydride (750 mg, 3.60 mmol) was added and the reaction was stirred at room temperature for 16 h. The suspension was concentrated under vacuum and the product was purified by Prep-LCMS to give 303 mg (65%) of N-(2-benzo[1,3]dioxol-5-yl-ethyl)-AP-(3-imidazol-1-yl-[1,2,4]thiadiazol-5-yl)-N,N-dimethyl-ethane-1,2-diamine. [M+H]⁻387.31; ¹H NMR (400 MHz, CDCl₃) δ 8.26 (s, 1H), 7.62 (s, 1H), 7.05 (s, 1H), 6.66-6.53 (m, 3H), 5.85 (s, 1H), 3.12(m, 3H), 2.68-2.60 (m, 6H), 2.31 (s, 3H).

### EXAMPLE 64

### N′-Benzo[1,3]dioxol-5-ylmethyl-N-methyl-N-(3-[1,2,4]triazol-1-yl-[1,2,4]thiadiazol-5-yl)-propane-1,3-diamine

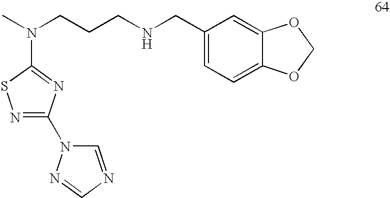 Example 64 was prepared following the procedures described in the preparation of Example 1 using sodium 1,2,4-triazole. [M+H]⁺374.12; ¹H-NMR (400 MHz, CDCl₃) δ 8.91 (s, 1H), 8.06 (s, 1H), 6.84 (s, 1H), 6.74 (d, 1H), 6.69 (d, 1H), 5.91 (s, 2H), 3.85 (s, 3 H), 3.64 (br s, 1H), 3.09 (br s, 2H), 2.82 (t, 2H), 2.05 (m, 2H), 1.99 (s, 2 H).

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method for the preparation of a compound of structural formula (III): 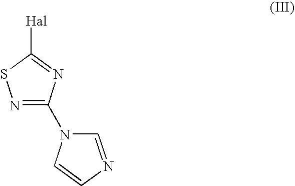
or a salt, ester, or prodrug thereof, wherein:
Hal is selected from the group consisting of chloro, bromo, and iodo; comprising the steps of:
a) treating a 3-amino-5-halo-[1,2,4]thiadiazole derivative of structural formula (II), 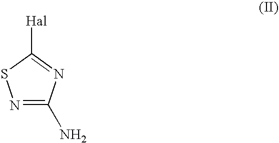
with a combination of suitable reagents, comprising a formaldehyde equivalent, a glyoxal equivalent, a ammonia equivalent, and an appropriate amount of a protic acid in a suitable protic solvent, using an appropriate reaction time over a suitable temperature range; and
b) isolating the novel reaction product of structural formula (III).

2. The method as recited in claim 1, wherein said formaldehyde equivalent is formalin or paraformaldehyde, either of which may be present in an amount greater than or equal to a stoichiometric amount.

3. The method as recited in claim 2, wherein paraformaldehyde is utilized as a reagent.

4. The method as recited in claim 3, wherein said paraformaldehyde is present in stoichiometric amounts.

5. The method as recited in claim 1, wherein said glyoxal equivalent is anhydrous glyoxal or glyoxal hydrate, either of which may be present in an amount greater than or equal to a stoichiometric amount.

6. The method as recited in claim 5, wherein glyoxal hydrate is employed as a reagent.

7. The method as recited in claim 6, wherein said glyoxal hydrate is present in stoichiometric amounts.

8. The method as recited in claim 1, wherein said ammonia equivalent is selected from the group consisting of ammonium chloride, ammonia gas, ammonium hydroxide, ammonium acetate, ammonium sulfate, ammonium bicarbonate, and ammonium carbamate, any of which may be present in an amount greater than or equal to a stoichiometric amount.

9. The method as recited in claim 8, which employs ammonium chloride as a reagent.

10. The method as recited in claim 9, wherein said ammonium chloride is present in stoichiometric 5 amounts.

11. The method as recited in claim 1, wherein said protic acid is phosphoric acid, present in an amount ranging from catalytic to greater than or equal to a stoichiometric amount.

12. The method as recited in claim 11, wherein said phosphoric acid is present in stoichiometric amounts.

13. The method as recited in claim 11, wherein said phosphoric acid is present in catalytic amounts.

14. The method as recited in claim 1, wherein:
said protic solvent is selected from the group consisting of ethanol, methanol, propanol, iso-propanol, butanol, tert-butanol, methoxyethanol, ethoxyethanol, ethylene glycol, and propylene glycol; or any of these protic solvents may be combined together and utilized as co-solvents.

15. The method as recited in claim 14, wherein ethanol is employed as the protic solvent.

16. The method as recited in claim 1, wherein the suitable temperature range is from about −20° C. to 150° C.

17. The method as recited in claim 16, wherein said suitable temperature range is from 40° C. to 90° C.

18. The method as recited in claim 1, wherein said reaction time range is from about 5 minutes to 48 hours.

19. The method as recited in claim 18, wherein said reaction time range is from 16 to 20 hours.

20. A method for the preparation of a compound of structural formula (V): 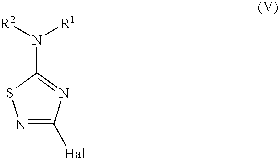
or a salt, ester, or prodrug thereof, wherein:
R¹ and R² may be independently selected from the group consisting of hydrogen, acyl, alkanoyl, alkenyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted; or R¹ and R² may be joined together to form a heterocycloalkyl ring; and
Hal is selected from the group consisting of chloro, bromo, and iodo; comprising:
a) treating a 3,5-dihalo-[1,2,4]thiadiazole of structural formula (IV), 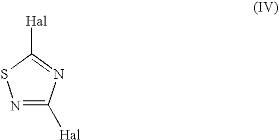
with an appropriate amine derivative R¹NHR², in a suitable aprotic solvent, using an appropriate reaction time over a suitable temperature range; and
b) optionally isolating the novel reaction product of structural formula (V).

21. The method as recited in claim 20, wherein:
said aprotic solvent is selected from the group consisting of dichloromethane, dimethyl sulfoxide, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H)-pyrimidinone, hexamethylphosphoramide, acetonitrile, pyridine, 2,6-lutidine, and 2,4,6-collidine; or any of these may be combined together and utilized as co-solvents.

22. The method as recited in claim 21, wherein dichloromethane is employed as the aprotic solvent.

23. The method as recited in claim 20, wherein a suitable temperature range for this reaction is from about −70° C. to 100° C.

24. The method as recited in claim 23, wherein the suitable temperature range is from −10° C. to 40° C.

25. The method as recited in claim 20, wherein a suitable time period for this reaction step ranges from about 5 minutes to 48 hours.

26. The method as recited in claim 25, wherein the suitable time period range is from 16 to 20 hours.

27. A method for the preparation of a compound of structural formula (I): 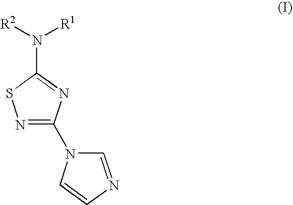
or a salt, ester, or prodrug thereof, wherein:
R¹ and R² may be independently selected from the group consisting of hydrogen, acyl, alkanoyl, alkenyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted; or R¹ and R² may also be taken together to form a heterocycloalkyl ring; comprising:
a) a reacting a compound of structural formula (III) with an appropriate amine derivative R¹NHR², in a suitable aprotic solvent, using an appropriate reaction time over a suitable temperature range, and
b) isolating the novel reaction product of structural formula (I) in high purity.

28. The method as recited in claim 27, wherein:
said aprotic solvent is selected from the group consisting of dichloromethane, dimethyl sulfoxide, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H)-pyrimidinone, hexamethylphosphoramide, acetonitrile, pyridine, 2,6-lutidine, and 2,4,6-collidine; or any of these may be combined together and utilized as co-solvents.

29. The method as recited in claim 28, wherein dichloromethane is employed as the aprotic solvent.

30. The method as recited in claim 27, wherein a suitable temperature range is from about −70° C. to 100° C.

31. The method as recited in claim 30, wherein the suitable temperature range is from −10° C. to 40° C.

32. The method as recited in claim 27, wherein a suitable time period ranges from about 5 minutes to 48 hours.

33. The method as recited in claim 32, wherein the suitable time period range is from 16 to 20 hours.

34. A method for the preparation of a compound of structural formula (I): 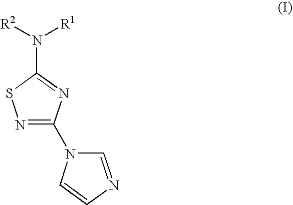
or a salt, ester, or prodrug thereof, wherein:
R¹ and R² may be independently selected from the group consisting of hydrogen, acyl, alkanoyl, alkenyl, alkoxy, alkoxyalkyl, alkyl, alkylaminocarbonyl, alkylsulfonyl, alkynyl, amido, amidoalkyl, amino, aroyl, aryl, arylalkyl, arylalkenyl, arylalkynyl, aryloxyarylalkyl, arylsulfonyl, arylalkylsulfonyl, arylalkenylsulfonyl, carbamoyl, carboalkoxy, carboalkoxyamino, cycloalkyl, cycloalkylalkyl, haloalkyl, heteroaroyl, heteroaryl, heteroarylalkyl, heteroarylalkenyl, heteroarylalkynyl, heteroarylsulfonyl, heteroarylalkylsulfonyl, heteroarylalkenylsulfonyl, heteroalkyl, heterocycloalkyl, hydroxyalkyl, perhaloalkyl, and trisubstituted silyl, any of which may be optionally substituted; or R¹ and R² may also be taken together to form a heterocycloalkyl ring; comprising:
a) treating a compound of structural formula (V) with a salt derivative of imidazole or a 1,2,4-triazole in a suitable dipolar aprotic solvent; using an appropriate reaction time over a suitable temperature range; and
b) isolating the desired product, in high purity.

35. The method as recited in claim 34 wherein:
said dipolar aprotic solvents are selected from the group consisting of dimethyl sulfoxide, sulfolane, N,N-dimethylformamide, N,N-dimethylacetamide, 1,3-dimethyl-3,4,5,6-tetrahydro-2(1H-pyrimidinone, and hexamethylphosphoramide; or any of these may be combined together and utilized as co-solvents.

36. The method as recited in claim 35, wherein dimethyl sulfoxide is employed as the dipolar aprotic solvent.

37. The method as recited in claim 34, wherein said salt derivative is selected from the group consisting of sodium, potassium, lithium and cesium.

38. The method as recited in claim 37, wherein said salt is the sodium derivative.

39. The method as recited in claim 34, wherein the suitable temperature range is from about −10° C. to 200° C.

40. The method as recited in claim 39, wherein a suitable temperature range is from 0° C. to 90° C.

41. The method as recited in claim 34, wherein a suitable time period range is from about 5 minutes to 48 hours.

42. The method as recited in claim 41, wherein the suitable time period range is from 8 to 16 hours.

43. A method for the preparation of a compound of structural formula (VI): 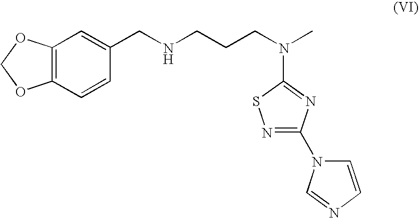
comprising:
a) reacting the compound of structural formula (IV), wherein Hal=Cl, with benzo[1,3]dioxol-5-ylmethyl-(3-methylamino-propyl)-carbamic acid tert-butyl ester in the presence of a trialkylamine in a suitable aprotic solvent, using an appropriate reaction time over a suitable temperature range, with or without isolating the reaction product of structural formula (VIa); 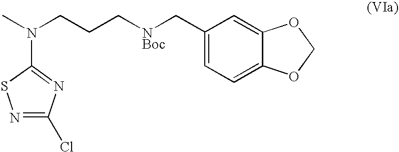
b) reacting the compound of structural formula (VIa) with a imidazole salt in a suitable dipolar aprotic solvent, using an appropriate reaction time over a suitable temperature range, and isolating the desired intermediate of structural formula (VIb): 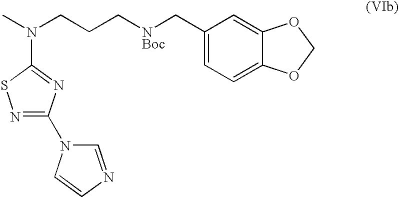
and
c) removing the Boc protecting group of structural intermediate (VIb) using trifluoroacetic acid in dichloromethane, followed by treatment with aqueous potassium carbonate solution, and isolating the desired product of structural formula (VI) in high yield and in a state of high purity.

44. The method as recited in claim 43, wherein:
said trialkylamine is selected from the group consisting of triethylamine, N,N-diisopropylethylamine, 4-methylmorpholine, and N-methylpiperidine.

45. The method as recited in claim 44, wherein triethylamine is employed as the trialkylamine.

46. The method as recited in claim 43, step a), wherein:
said suitable aprotic solvents are selected from the group consisting of dichloromethane, dimethyl sulfoxide, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H)-pyrimidinone, hexamethylphosphoramide, acetonitrile, pyridine, 2,6-lutidine, and 2,4,6-collidine; or any of these may be combined together and utilized as co-solvents.

47. The method as recited in claim 46, wherein dichloromethane is employed as the aprotic solvent.

48. The method as recited in claim 43, step a), wherein a suitable temperature range is from about −70° C. to 100° C.

49. The method as recited in claim 48, wherein the suitable temperature range is from −10° C. to 40° C.

50. The method as recited in claim 43, step a), wherein said reaction time period ranges from about 5 minutes to 48 hours.

51. The method as recited in claim 50, wherein said reaction time period range is from 12 to 18 hours.

52. The method as recited in claim 43, step b), wherein:
said suitable dipolar aprotic solvents are selected from the group consisting of dimethyl sulfoxide, sulfolane, N,N-dimethylformamide, N,N-dimethylacetamide, 1,3-dimethyl-3,4,5,6-tetrahydro-2(1H-pyrimidinone, and hexamethylphosphoramide; or any of these may be combined together and utilized as co-solvents.

53. The method as recited in claim 52, wherein dimethyl sulfoxide is employed as the dipolar aprotic solvent.

54. The method as recited in claim 43, step b), wherein said imidazole salt derivative is selected from the group consisting of sodium, potassium, lithium and cesium.

55. The method as recited in claim 54, wherein said imidazole salt is the sodium derivative.

56. The method as recited in claim 43, step b), wherein the suitable temperature range is from about −20 10° C. to 200° C.

57. The method as recited in claim 56, wherein a suitable temperature range is from 10° C. to 90° C.

58. The method as recited in claim 43, step b), wherein a suitable time period range is from about 5 minutes to 48 hours.

59. The method as recited in claim 58, wherein the suitable time period range is from 8 to 16 hours.

60. A method for the preparation of a compound of structural formula (VI): 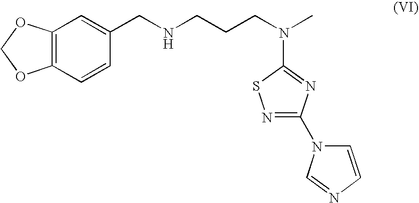
comprising:
a) reacting the compound of structural formula (III), 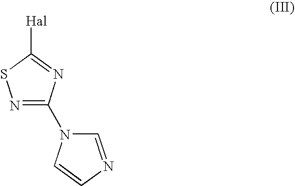
wherein Hal=Cl, with benzo[1,3]dioxol-5-ylmethyl-(3-methylamino-propyl)-carbamic acid tert-butyl ester in the presence of a trialkylamine in a suitable aprotic solvent, using an appropriate reaction time over a suitable temperature range, with or without isolating the reaction product of structural formula (VIb); 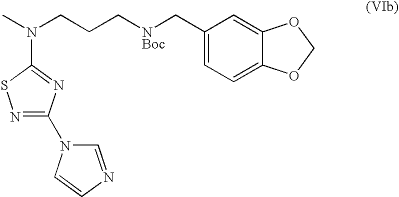
then:
b) removing the Boc protecting group of the compound of structural formula (VIb) using trifluoroacetic acid in dichloromethane, followed by treatment with aqueous potassium carbonate solution, and isolating the desired product of structural formula (VI) in high yield and in a state of high purity.

61. The method as recited in claim 60, wherein:
said trialkylamine is selected from the group consisting of triethylamine, N,N-diisopropylethylamine, 4-methylmorpholine, and N-methylpiperidine.

62. The method as recited in claim 61, wherein triethylamine is employed as the trialkylamine.

63. The method as recited in claim 60, wherein:
said suitable aprotic solvents are selected from the group consisting of dichloromethane, dimethyl sulfoxide, sulfolane, toluene, xylene, benzene, dichloroethane, chloroform, acetone, 2-butanone, ethyl acetate, 1,4-dioxane, tetrahydrofuran, dimethoxyethane, diethyl ether, tert-butyl methyl ether, N,N-dimethylformamide, N,N-dimethylacetamide, 1,3-dimethyl-3,4,5,6-tetrahydro-2(1H)-pyrimidinone, hexamethylphosphoramide, acetonitrile, pyridine, 2,6-lutidine, and 2,4,6-collidine; or any of these may be combined together and utilized as co-solvents.

64. The method as recited in claim 63, wherein dichloromethane is employed as the aprotic solvent.

65. The method as recited in claim 60, wherein a suitable temperature range is from about −70° C. to 100° C.

66. The method as recited in claim 65, wherein the suitable temperature range is from −10° C. to 40° C.

67. The method as recited in claim 60, wherein said reaction time period ranges from about 5 minutes to 48 hours.

68. The method as recited in claim 67, wherein said reaction time period range is from 12 to 18 hours.
